SEQUENCE LISTING (11 sequences)

SEQ ID NO: 1 - DNA, 426 bp, Homo sapiens
atggaggggacaatgatgatgcagcagaggccagttctgagccaacagcaccctagtttcattctcaactctagccctgcacactcacctatggcccgggagattgacaacttctaccctgagcgcttcacctaccacaatgtgcgcctctgggatgaggagtcggcccagctgctgccgcactggaaggagacgcaccgcttcattgaggctgcaagagcacagggcacccacgtgctggtccactgcaagatgggcgtcagccgctcagcggccacagtgctggcctatgccatgaagcagtacgaatgcagcctggagcaggccctgcgccacgtgcaggagctccggcccatcgcccgccccaaccctggcttcctgcgccagctgcagatctaccagggcatcctgacggccagaacctga

SEQ ID NO: 2 - protein, 141 aa, Homo sapiens
MEGTMMMQQRPVLSQQHPSFILNSSPAHSPMAREIDNFYPERFTYHNVRLWDEESAQLLPHWKETHRFIEAARAQGTHVLVHCKMGVSRSAATVLAYAMKQYECSLEQALRHVQELRPIARPNPGFLRQLQIYQGILTART

SEQ ID NO: 3 - protein, 384 aa, Homo sapiens
MKVTSLDGRQLRKMLRKEAAARCVVLDCRPYLAFAASNVRGSLNVNLNSVVLRRARGGAVSARYVLPDEAARARLLQEGGGGVAAVVVLDQGSRHWQKLREESAARVVLTSLLACLPAGPRVYFLKGGYETFYSEYPECCVDVKPISQEKIESERALISQCGKPVVNVSYRPAYDQGGPVEILPFLYLGSAYHASKCEFLANLHITALLNVSRRTSEACMTHLHYKWIPVEDSHTADISSHFQEAIDFIDCVREKGGKVLVHCEAGISRSPTICMAYLMKTKQFRLKEAFDYIKQRRSMVSPNFGFMGQLLQYESEILPSTPNPQPPSCQGEAAGSSLIGHLQTLSPDMQGAYCTFPASVLAPVPTHSTVSELSRSPVATATSC

SEQ ID NO: 4 - DNA, 1755 bp, Homo sapiens
tgtcctgcgggtccaggactgtccgcggggttgagggaaggggccgtgcccggtgccagcccaggtgctcgcggcctggctccatggccctggtcacagtgagccgttcgcccccgggcagcggcgcctccacgcccgtggggccctgggaccaggcggtccagcgaaggagtcgactccagcgaaggcagagctttgcggtgctccgtggggctgtcctgggactgcaggatggaggggacaatgatgatgcagcagaggccagttctgagccaacagcaccctagtttcattctcaactctagccctgcacactcacctatggcccgggagattgacaacttctaccctgagcgcttcacctaccacaatgtgcgcctctgggatgaggagtcggcccagctgctgccgcactggaaggagacgcaccgcttcattgaggctgcaagagcacagggcacccacgtgctggtccactgcaagatgggcgtcagccgctcagcggccacagtgctggcctatgccatgaagcagtacgaatgcagcctggagcaggccctgcgccacgtgcaggagctccggcccatcgcccgccccaaccctggcttcctgcgccagctgcagatctaccagggcatcctgacggccagaacctgagggtggtggggaggagaaggttgtaggcatggaagagagccaggcagccccgaaagaagagcctgggccacggccacgtataaacctccgaggggtcatgaggtccatcagtcttctggagccctccttggagctggagagcacctcagagaccagtgacatgccagaggtcttctcttcccacgagtcttcacatgaagagcctctgcagcccttcccacagcttgcaaggaccaagggaggccagcaggtggacagggggcctcagcctgccctgaagtcccgccagtcagtggttaccctccagggcagtgccgtggtggccaaccggacccaggccttccaggagcaggagcaggggcaggggcaggggcagggagagccctgcatttcctctacgcccaggttccggaaggtggtgagacaggccagcgtgcatgacagtggagaggagggcgaggcctgagccctcacacatgcccacgctcccctgacactgaagaggatccacaactccttggagaaacaccctcacgtctgttgccgcacacattcctctcagctccgccccatacccgtcactacagcctcacctcccacccctgtcactacggcctcacctcccacccctgtcactacagcctcacctcctacagccttaagtcccaggcccatgtctgcctgtccaagggctcaagactttctaactgggatgtggtagagggactgaaggtacctttgggggcaacagcaccctagtttcattctcaactctagccctgcacctcctgtcctctcccagttcattcctggaaccagccaggccaggcaaccagtggcccccaaaggcaggcaggatcctcaggccccagccgcgggaggctggaagggctggcagatcgcttccctcatccacctccaccggtccaggtctttgctgctgtccccagacctcctgtgacaccacgccagatcacagggcaccaggccagagatagtcttctttttgtcctttctggcctctggctagtcagtttttcatagccttacagtatctggctttgtactgagaaataaaacacattttcatatttggtt

SEQ ID NO: 5 - protein, 173 aa, Homo sapiens
GPSEILPHLYLGSYSTASEANLALLKKLGITHVINVTEEVPNPFELDKKNDRHYTNAYISKNSGFTYLQIPNVDDHIYYHIAWNHETKISKYFDEAVDFIDDARQKGGKVLVHCQAGISRSATLIIAYLMKTRNLSLNEAYDFVYVYHIKERRCPIISPNFGFLRQLIEYERK

SEQ ID NO: 6 - protein, 109 aa, Homo sapiens
VTREIDNFFPGTFEYFNVRVYDDEKTNLLKYWDDTFRYITRAKAEGSKVLVHCKMGVSRSASVVIAYAMKAYQWEFQQALEHVKKRRSCIKPNKNFLNQLETYSGMLDA

SEQ ID NO: 7 - DNA, 409 bp, Homo sapiens
  misc_feature (372)..(372): n=a, c, g or t
ggcgatccgtgctagctgtggaaagtgttggatgtcagtgacctggagagtgtcacttccaaagagatccgccaggctctggagctgcgcctggggctccccctccagcagtaccgtgacttcatcgacaaccagatgctgctgctggtggcacagcgggaccgagcctcccgcatcttcccccacctctacctgggctcagagtggaacgcagcaaacctggaggagctgcagaggaacagggtcacccacatcttgaacatggcccgggagattgacaacttctaccctgagcgcttcacctaccacaatgtgcgcctctgggatgaggagtcggcccagctgctgccgcactggaaggagacgcaccgnttcattgaggctgcaagagcacagggcacccacgtgc

SEQ ID NO: 8 - DNA, 599 bp, Homo sapiens
tggaacgcagcaaacctggaggagctgcagaggaacagggtcacccacatcttgaacatggcccgggagattgacaacttctaccctgagcgcttcacctaccacaatgtgcgcctctgggatgaggagtcggcccagctgctgccgcactggaaggagacgcaccgcttcattgaggctgcaagagcacagggcacccacgtgctggtccactgcaagatgggcgtcagccgctcagcggccacagtgctggcctatgccatgaagcagtacgaatgcagcctggagcaggccctgcgccacgtgcaggagctccggcccatcgcccgccccaaccctggcttcctgcgccagctgcagatctaccagggcatcctgacggccagaacctgagggtggtggggaggagaaggttgtaggcatggaagagagccaggcagccccgaaagaagagcctgggccacggccacgtataaacctccgaggggtcatgaggtccatcagtcttctggagccctccttggagctggagagcacctcagagaccagtgacatgccagaggtcttctcttcccacgagtcttcacatgaagagcctc

SEQ ID NO: 9 - DNA, 494 bp, Homo sapiens
cgcactggaaggagacgcaccgcttcattgaggctgcaagagcacagggcacccacgtgctggtccactgcaagatgggcgtcagccgctcagcggccacagtgctggcctatgccatgaagcagtacgaatgcagcctggagcaggccctgcgccacgtgcaggagctccggcccatcgcccgccccaaccctggcttcctgcgccagctgcagatctaccagggcatcctgacggccagccgccagagccatgtctgggagcagaaagtgggtggggtctccccagaggagcacccagcccctgaagtctctacaccattcccacctcttccgccagaacctgagggtggtggcgtcggggggggccccgctcccaattcgcccttatttgagtttgattacaaaaaattggcccgttttttacaaactttttgcctgggaaaaccctggggtttacgcaacatattggaatttggaccaatatcctctttt

SEQ ID NO: 10 - DNA, 2322 bp, Homo sapiens
aggggcgtgcccggtgcagcccaggtgctcgcgtgctggctcatggccctgtgtcacagtgagccgttcgcccccgtggcagctggcgcctccaccgccctgtggggccctggaattcctgagagaggggaggggacagccctccccgccctcaccctggggctgctctctcggcaggacctggtccagcgaaggagtcgactccagcgaagagctttgcggtgctcctgtggggctgtcctgggactgcaggatggaggggacaatgatgatgcagcagaggccagttctgagccaacagagaaggccccgagtgaggaggagctccacgggggaacccagacagactttcggtgcaaggatcccagagtccccagaagcaggaggagcagaggcagcaactgcaacctcatgaggcgtgctgaggccgcagggatgacatccgacttggaagcccaagctggaggcacccccgggctccccgggatccggataccttgcttggtagtttctacacgagaagggagaaggtctgagcccaggatgagacggtcctcctgggacgtggatttccctgacagcagctcccccagctgcaccctgggcctggtcttgcccctctggagtgacacccaggtgtacttatatggagacgggggcttcagcgtgacgtctggtgggcaaagccggatcttcaagcccatctccatccagaccatgtgggccacactccaggtattgcaccaagcatgtgaggcagctctaggcagcggccttgtaccgggtggcagtgccctcacctgggccagccactaccaggagagactgaactccgaacagagctgcctcaatgagtggacggctatggccgacctggagtctctgcggcctcccagcgccgagcctggcgggtcctcagaacaggagcagatggagcgggcgatccgtgctgagctgtggaaagtgttggatgtcggtgacctggagagtgtcgcttccagagagatccgccaggctctggagctgcgcctggggctccccctccagcagtaccgtgacttcatcgacaaccagatgctgctgctggtggcacagcgggaccgagcctcccgcatcttcccccacctctacctgggctcagagtggaacgcagcaaacctggaggagctgcagaggaacagggtcacccacatcttgatggcccgggagattgacaacttctaccctgagcgcttcacctaccacaatgtgcgcctctgggatgaggagtcggcccagctgctgccgcactggaaggagacgcaccgcttcattgaggctgcaagagcacagggcacccacgtgctggtccactgcaagatgggcgtcagccgctcagcggccacagtgctggcctatgccatgaagcagtacgaatgcagcctggagcaggccctgcgccacgtgcaggagctccggcccatcgcccgccccaaccctggcttcctgcgccagctgcagatctaccagggcatcctgacggccagccgccagagccatgtctgggagcagaaagtgggtggggtctccccagaggagcacccagcccctgaagtctctacaccattcccacctcttccgccagaacctgagggtggtggggaggagaaggttgtaggcatggaagagagccaggcagccccgaaagaagagcctgggccacggccacgtataaacctccgaggggtcatgaggtccatcagtcttctggagccctccttggagctggagagcacctcagagaccagtgacatgccagaggtcttctcttcccacgagtcttcacatgaagagcctctgcagcccttcccacagcttgcaaggaccaagggaggccagcaggtggacagggggcctcagcctgccctgaagtcccgccagtcagtggttaccctccagggcagtgccgtggtggccaaccggacccaggccttccaggagcaggagcaggggcaggggcaggggcagggagagccctgcatttcctctacgcccaggttccggaaggtggtgagacaggccagcgtgcatgacagtggagaggagggcggcctgagccctcacacatgcccacgctcccctgacactgaagaggatccacaactccttggagaaacaccctcacgtctgttgccgcacacattcctctcagctccgccccatacccgtcactacagcctcacctcccacccctgtcactacggcctcacctcccacccctgtcactacagcctcacctcctacagccttaagtcccaggcccatgtctgcctgtccaagggc

SEQ ID NO: 11 - protein, 779 aa, Homo sapiens
  MISC_FEATURE (701)..(701): Xaa=any amino acid
RGVPGAAQVLACWLMALCHSEPFAPVAAGASTALWGPGIPERGEGTALPALTALGLLSRQDRLVQRRSRLQRRALRCSCGAVLGLQDGGDNDDAAEASSEPTEKAPSEEELHGGTQTDFRCKDPRVPRSRRSRGSNCNLMVRRAEAAGMTSDLEAQAGGTPGLPGIRIPCLVVSTREGRRSEPRMRRSSWDVDFPDSSSPSCTLGLVLPLWSDTQVYLYGDGGFSVTSGGQSRIFKPISIQTMWATLQVLHQACEAALGSGLVPGGSALTWASHYQERLNSEQSCLNEWTAMADLESLRPPSAEPGGSSEQEQMERAIRAELWKVLDVGDLESVASREIRQALELRLGLPLQQYRDFIDNQMLLLVAQRDRASRIFPHLYLGSEWNAANLEELQRNRVTHILYMAREIDNFYPERFTYHNVRLWDEESAQLLPHWKETHRFIEAARAQGTHVLVHCKMGVSRSAATVLAYAMKQYECSLEQALRHVQELRPIARPNPGFLRQLQIYQGILTASRQSHVWEQKVGGVSPEEHPAPEVSTPFPPLPPEPEGGGEEKVVGMEESQAAPKEEPGPRPRINLRGVMRSISLLEPSLELESTSETSDMPEVFSSHESSHEEPLQPFPQLARTKGGQQVDRGPQPALKSRQSVVTLQGSAVVANRTQAFQEQEQGQGQGQGEPCISSTPRFRKVVRQASVHDSGEEGXGLSPHTCPRSPDTEEDPQLLGETPSRLLPHTFLSAPPHTRHYSLTSHPCHYGLTSHPCHYSLTSYSLKSQAHVCLSKG

(19) United States
(12) Patent Application Publication
(10) Pub. No.: US 2003/0170856 A1
(43) Pub. Date: Sep. 11, 2003

(54) Regulation of human map kinase phosphatase-like enzyme

(76) Inventor: Jiing-Ren Liou, Belmont, MA (US)

Correspondence Address: BANNER & WITCOFF, 1001 G STREET N W, SUITE 1100, WASHINGTON, DC, 20001, US

(21) Appl. No.: 10/363,676

(22) PCT Filed: Aug. 27, 2001
(86) PCT No.: PCT/EP01/09848

(51) Int. Cl. C12Q 1/68; C07H 21/04; C12N 9/12; C12N 9/16; C12P 21/02; C12N 5/06

(52) U.S. Cl. 435/196; 435/194; 435/320.1; 435/325; 435/6; 536/23.2; 435/69.1

(57) ABSTRACT

Reagents which regulate human MAP kinase phosphatase-like enzyme and reagents which bind to human MAP kinase phosphatase-like enzyme gene products can play a role in preventing, ameliorating, or correcting dysfunctions or diseases including, but not limited to, allergies including asthma, CNS disorders, diabetes, obesity, chronic obstructive pulmonary disease, cancer, and cardiovascular diseases.

### TECHNICAL FIELD OF THE INVENTION

The invention relates to the area of enzyme regulation. More particularly, the invention relates to the regulation of human MAP kinase phosphatase-like enzyme and its regulation.

### BACKGROUND OF THE INVENTION

Mitogen-activated protein kinases (MAP kinases) mediate multiple cellular pathways regulating growth (1) and differentiation (2, 3, U.S. Pat. No. 5,998,188). In neuronal cells, MAP kinase activity mediates the actions of growth factors like EGF that stimulate cellular proliferation as well as factors like NGF that maintain neuronal survival and differentiation (4-6). Such ligand-activated signal transduction pathways involve activation of receptor tyrosine kinases which initiates a series of phosphorylation events that activate a cascade of serine/threonine kinases converging on the MAP kinase (also called extracellular signal regulated kinase (ERK)) isoforms, ERK1 and ERK2 (7-9).

Activation of MAP kinase involves specific phosphorylations on threonine and tyrosine residues within the Thr-Glu-Tyr motif (10) by MAP kinase kinase (MAP kinase and ERK kinase or MEK) (2, 11). Phosphorylation of both these residues is required for MAP kinase activation (11, 12). It has been suggested that the inactivation of MAP kinase is a critical event that regulates the physiological response to MAP kinase activation (13). This inactivation is mediated, in part, by dephosphorylation of MAP kinases by dual specificity phosphatases called MKPs (MAP kinase phosphatases) that dephosphorylate both the threonine and tyrosine residues phosphorylated by MEK (13-16). The activation of MAP kinase appears to be tightly regulated through the coordinate action of MEK and MKPs. By regulating the extent of MAP kinase activation, these MKPs may dictate the choice of differentiation or proliferation within a developing cell (17).

The prototype dual-specificity phosphatase, VH1, was identified in vaccinia and showed similarity to cdc25, a protein that controls cell entry into mitosis (18). VH1 homologues from human (PAC-1, CL100, and most recently B23), mouse [MKP-1 (3CH134 or erp)], and yeast (Yop51, MSG5) have also been isolated (19-24.) All are dual-specificity phosphatases that specifically dephosphorylate MAP kinase in vitro (25) and in vivo (13, 15, 26). MKP-1 (also called 3CH134 or erp) was discovered as an immediate early gene whose rapid transcription and subsequent translation are suggested to provide a feed-back loop to terminate growth factor signals (13, 19, 26). Overexpression of mouse MKP-1 was shown to inhibit dramatically fibroblast proliferation suggesting that the inactivation of MAP kinase in vivo by MKP-1 has a profound negative effect on cellular proliferation (25, 26).

MAP kinase activation by growth factors has been extensively studied in PC12 cells (27). PC12 cells originate from a rat pheochromocytoma and retain many features of neural crest-derived cells, most notably the ability to undergo neuronal differentiation upon stimulation by NGF (28). Transfection with activated forms of the oncogenes ras, raf-1 and src into PC12 cells is sufficient for differentiation in the absence of NGF stimulation (6, 8, 29). As each of these genes has been shown to converge on MAP kinase activation, this implies that components of the MAP kinase cascade are required for neuronal differentiation. More recently it has been shown that the activation of MAP kinase kinase, MAPKK-1, is required and sufficient for PC12 cell differentiation (3). Despite our understanding of MAP kinase activation in neuronal differentiation, we know relatively little about MAP kinase inactivation.

Because of the important role of MAP kinase phosphatases in cell growth and differentiation, it is desirable to have molecular tools useful for regulation of these enzymes. The present invention presents such tools.

### SUMMARY OF THE INVENTION

It is an object of the invention to provide reagents and methods of regulating a human MAP kinase phosphatase-like enzyme. This and other objects of the invention are provided by one or more of the embodiments described below.

One embodiment of the invention is a MAP kinase phosphatase-like enzyme polypeptide comprising an amino acid sequence selected from the group consisting of:

amino acid sequences which are at least about 50% identical to the amino acid sequence shown in SEQ ID NO: 2;

the amino acid sequence shown in SEQ ID NO: 2;

amino acid sequences which are at least about 50% identical to the amino acid sequence shown in SEQ ID NO: 11; and

the amino acid sequence shown in SEQ ID NO: 11.

Yet another embodiment of the invention is a method of screening for agents which decrease extracellular matrix degradation. A test compound is contacted with a MAP kinase phosphatase-like enzyme polypeptide comprising an amino acid sequence selected from the group consisting of:

amino acid sequences which are at least about 50% identical to the amino acid sequence shown in SEQ ID NO: 2;

the amino acid sequence shown in SEQ ID NO: 2;

amino acid sequences which are at least about 50% identical to the amino acid sequence shown in SEQ ID NO: 11; and

the amino acid sequence shown in SEQ ID NO:11.

Binding between the test compound and the MAP kinase phosphatase-like enzyme polypeptide is detected. A test compound which binds to the MAP kinase phosphatase-like enzyme polypeptide is thereby identified as a potential agent for decreasing extracellular matrix degradation. The agent can work by decreasing the activity of the MAP kinase phosphatase-like enzyme.

Another embodiment of the invention is a method of screening for agents which decrease extracellular matrix degradation. A test compound is contacted with a polynucleotide encoding a MAP kinase phosphatase-like enzyme polypeptide, wherein the polynucleotide comprises a nucleotide sequence selected from the group consisting of:

nucleotide sequences which are at least about 50% identical to the nucleotide sequence shown in SEQ ID NO: 1;

the nucleotide sequence shown in SEQ ID NO: 1;

amino acid sequences which are at least about 50% identical to the amino acid sequence shown in SEQ ID NO: 10; and

the amino acid sequence shown in SEQ ID NO:10.

Binding of the test compound to the polynucleotide is detected. A test compound which binds to the polynucleotide is identified as a potential agent for decreasing extracellular matrix degradation. The agent can work by decreasing the amount of the

MAP kinase phosphatase-like enzyme through interacting with the MAP kinase phosphatase-like enzyme mRNA.

Another embodiment of the invention is a method of screening for agents which regulate extracellular matrix degradation. A test compound is contacted with a MAP kinase phosphatase-like enzyme polypeptide comprising an amino acid sequence selected from the group consisting of:

amino acid sequences which are at least about 50% identical to the amino acid sequence shown in SEQ ID NO: 2;

the amino acid sequence shown in SEQ ID NO: 2;

amino acid sequences which are at least about 50% identical to the amino acid sequence shown in SEQ ID NO: 11; and

the amino acid sequence shown in SEQ ID NO:11.

A MAP kinase phosphatase-like enzyme activity of the polypeptide is detected. A test compound which increases MAP kinase phosphatase-like enzyme activity of the polypeptide relative to MAP kinase phosphatase-like enzyme activity in the absence of the test compound is thereby identified as a potential agent for increasing extracellular matrix degradation. A test compound which decreases MAP kinase phosphatase-like enzyme activity of the polypeptide relative to MAP kinase phosphatase-like enzyme activity in the absence of the test compound is thereby identified as a potential agent for decreasing extracellular matrix degradation.

Even another embodiment of the invention is a method of screening for agents which decrease extracellular matrix degradation. A test compound is contacted with a MAP kinase phosphatase-like enzyme product of a polynucleotide which comprises a nucleotide sequence selected from the group consisting of:

nucleotide sequences which are at least about 50% identical to the nucleotide sequence shown in SEQ ID NO: 1;

the nucleotide sequence shown in SEQ ID NO: 1;

amino acid sequences which are at least about 50% identical to the amino acid sequence shown in SEQ ID NO: 10; and

the amino acid sequence shown in SEQ ID NO:10.

Binding of the test compound to the MAP kinase phosphatase-like enzyme product is detected. A test compound which binds to the MAP kinase phosphatase-like enzyme product is thereby identified as a potential agent for decreasing extracellular matrix degradation.

Still another embodiment of the invention is a method of reducing extracellular matrix degradation. A cell is contacted with a reagent which specifically binds to a polynucleotide encoding a MAP kinase phosphatase-like enzyme polypeptide or the product encoded by the polynucleotide, wherein the polynucleotide comprises a nucleotide sequence selected from the group consisting of:

nucleotide sequences which are at least about 50% identical to the nucleotide sequence shown in SEQ ID NO: 1;

the nucleotide sequence shown in SEQ ID NO: 1;

amino acid sequences which are at least about 50% identical to the amino acid sequence shown in SEQ ID NO: 10; and

the amino acid sequence shown in SEQ ID NO:10.

MAP kinase phosphatase-like enzyme activity in the cell is thereby decreased.

The invention thus provides a human MAP kinase phosphatase-like enzyme which can be used to identify test compounds which may act, for example, as agonists or antagonists at the enzyme's active site. Human MAP kinase phosphatase-like enzyme and fragments thereof also are useful in raising specific antibodies which can block the enzyme and effectively reduce its activity.

### BRIEF DESCRIPTION OF THE DRAWINGS

FIG. 1 shows the DNA-sequence encoding a MAP kinase phosphatase-like enzyme polypeptide (SEQ ID NO:1).

FIG. 2 shows the amino acid sequence deduced from the DNA-sequence of FIG. 1 (SEQ ID NO:2).

FIG. 3 shows the amino acid sequence of the protein identified by SwissProt Accession No. Q16690 (SEQ ID NO:3).

FIG. 4 shows the DNA-sequence encoding a MAP kinase phosphatase-like enzyme polypeptide (SEQ ID NO: 4).

FIG. 5 shows the amino acid sequence of pfamm/hmm/DSPc (SEQ ID NO:6).

FIG. 6 shows the amino acid sequence of trembl/AB036834/AB036834_{—}1 product (SEQ ID NO:7).

FIG. 7 shows the DNA-sequence encoding a MAP kinase phosphatase-like enzyme polypeptide (SEQ ID NO:8).

FIG. 8 shows the DNA-sequence encoding a MAP kinase phosphatase-like enzyme polypeptide (SEQ ID NO:9).

FIG. 9 shows the DNA-sequence encoding a MAP kinase phosphatase-like enzyme polypeptide (SEQ ID NO:10).

FIG. 10 shows the BLASTP alignment of human MAP kinase phosphatase-like enzyme (SEQ ID NO:2) against trembl|AB036834|AB036834_{—}1 product: “MAP kinase phosphatase” (Drosophila melanogaster mRNA for MAP kinase phosphatase, complete; SEQ ID NO:7).

FIG. 11 shows the HMMPFAM alignment of human MAP kinase phosphatase-like enzyme against pfam|hmm|DSPc (SEQ ID NO:6).

FIG. 12 shows the BLOCKS search results.

FIG. 13 shows the DNA sequence encoding a MAP kinase phosphatase-like enzyme polypeptide (SEQ ID NO: 10).

FIG. 14 shows the amino acid sequence deduced from the DNA sequence of FIG. 13 (SEQ ID NO:11).

FIG. 15 shows the BLAST alignment of 184 protein against trembl/AB036834/AB036834_{—}1.

FIG. 16 shows the HMMPFAM alignment of 184 protein against pfam/hmm/DSPc.

FIG. 17 shows the results of the expression profiling of MAP kinase phosphatase-like mRNA in the whole body screen

FIG. 18 shows the results of the expression profiling of MAP kinase phosphatase-like mRNA in the blood/lung screen

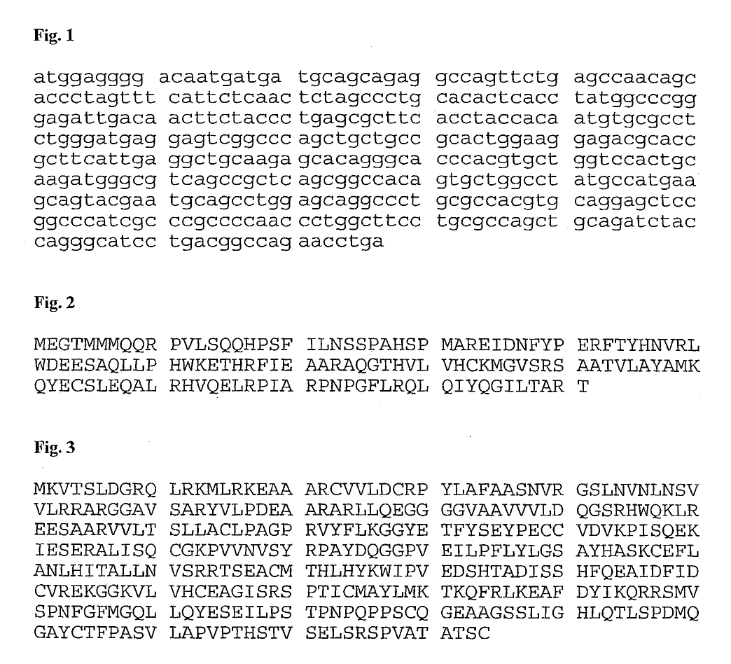

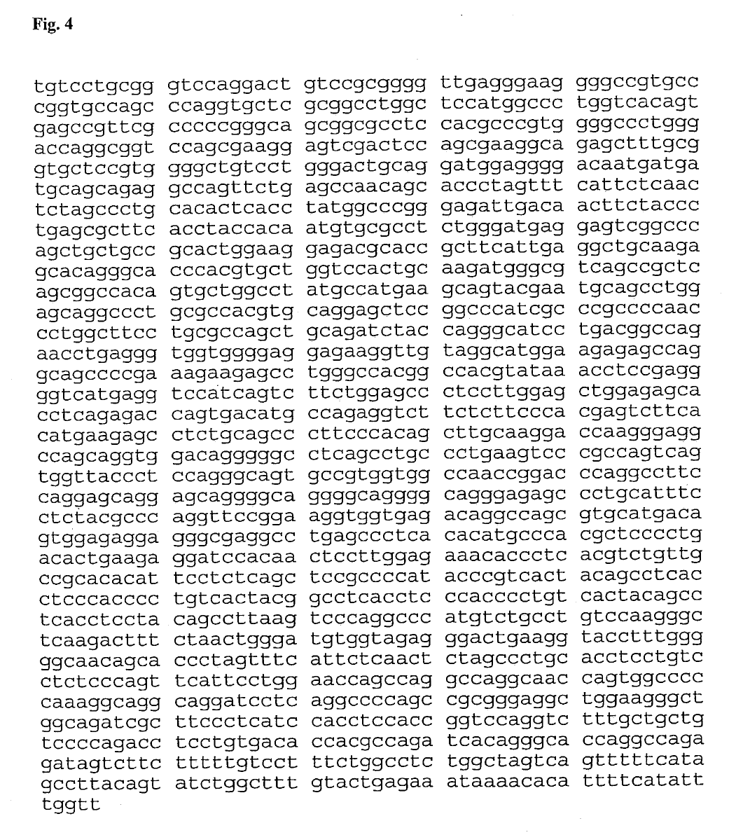

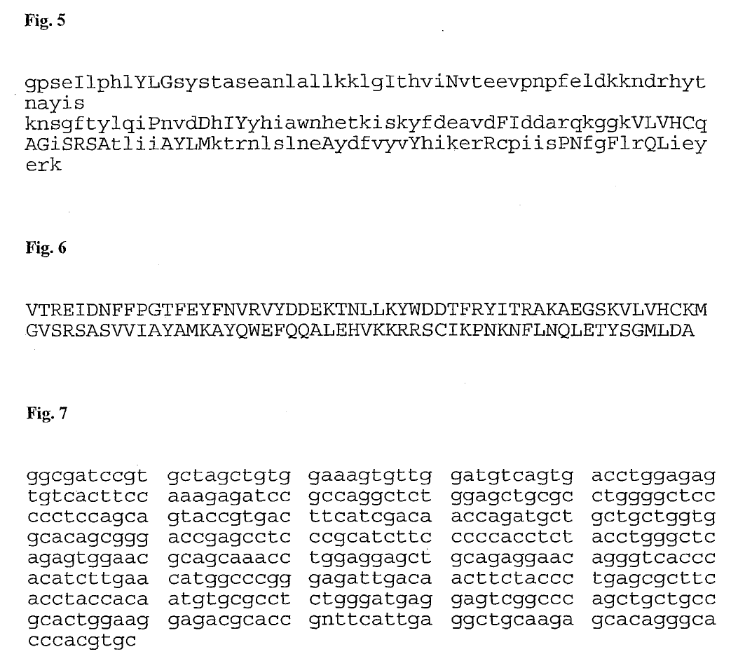

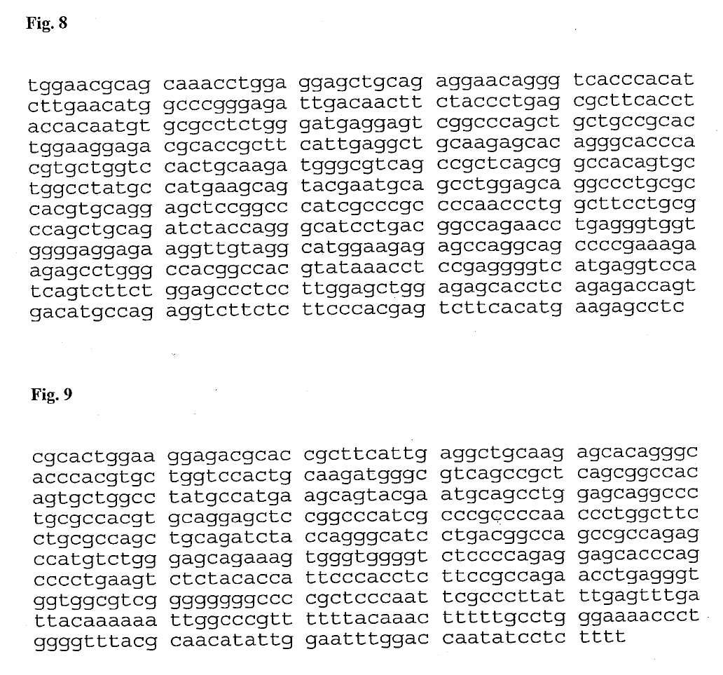

### DETAILED DESCRIPTION OF THE INVENTION

The invention relates to an isolated polynucleotide encoding a MAP kinase phosphatase-like enzyme polypeptide and being selected from the group consisting of:

a) a polynucleotide encoding a MAP kinase phosphatase-like enzyme poly-peptide comprising an amino acid sequence selected from the group consisting of:

amino acid sequences which are at least about 50% identical to

the amino acid sequence shown in SEQ ID NO: 2;

the amino acid sequence shown in SEQ ID NO: 2;

amino acid sequences which are at least about 50% identical to

the amino acid sequence shown in SEQ ID NO: 11; and

the amino acid sequence shown in SEQ ID NO:11.

b) a polynucleotide comprising the sequence of SEQ ID NO: 1 or 10;

c) a polynucleotide which hybridizes under stringent conditions to a polynucleotide specified in (a) and (b);

d) a polynucleotide the sequence of which deviates from the polynucleotide sequences specified in (a) to (c) due to the degeneration of the genetic code; and

e) a polynucleotide which represents a fragment, derivative or allelic variation of a polynucleotide sequence specified in (a) to (d).

Furthermore, it has been discovered by the present applicant that a novel MAP kinase phosphatase-like enzyme, particularly a human MAP kinase phosphatase-like enzyme, is a discovery of the present invention. Human MAP kinase phosphatase-like enzyme comprises the amino acid sequence shown in SEQ ID NO:2 or 11. Human MAP kinase phosphatase-like enzyme was identified by searching human sequences with the human protein having the sequence shown in SEQ ID NO:3 and identified with SwissProt Accession No. Q16690.

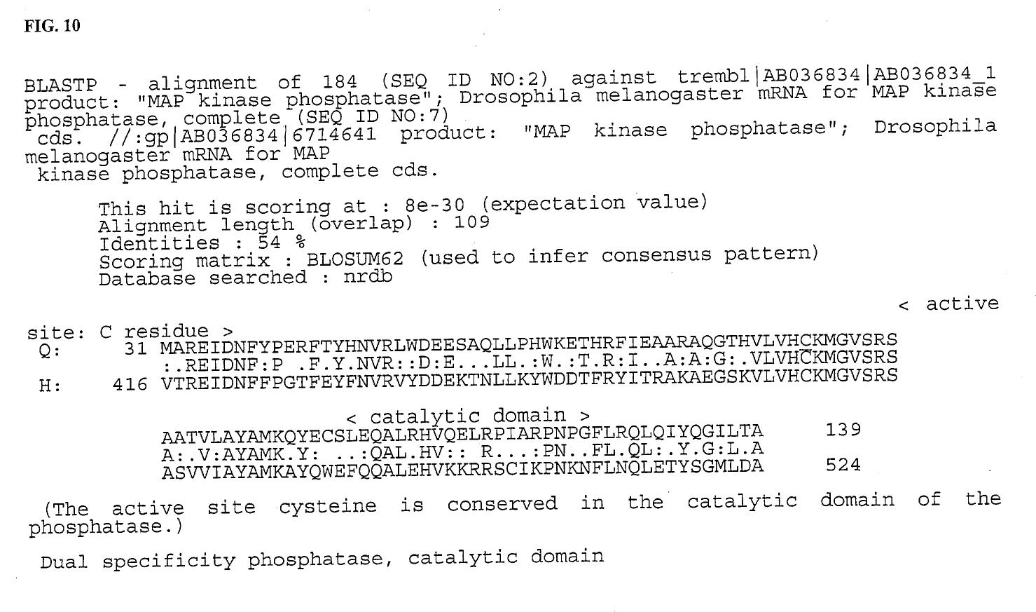

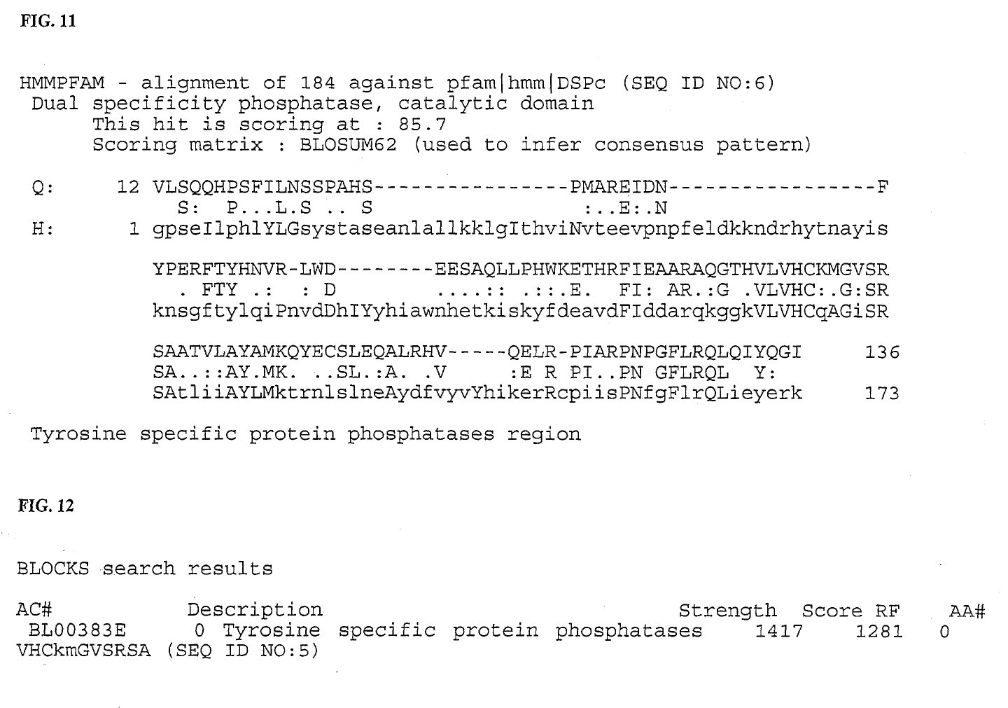

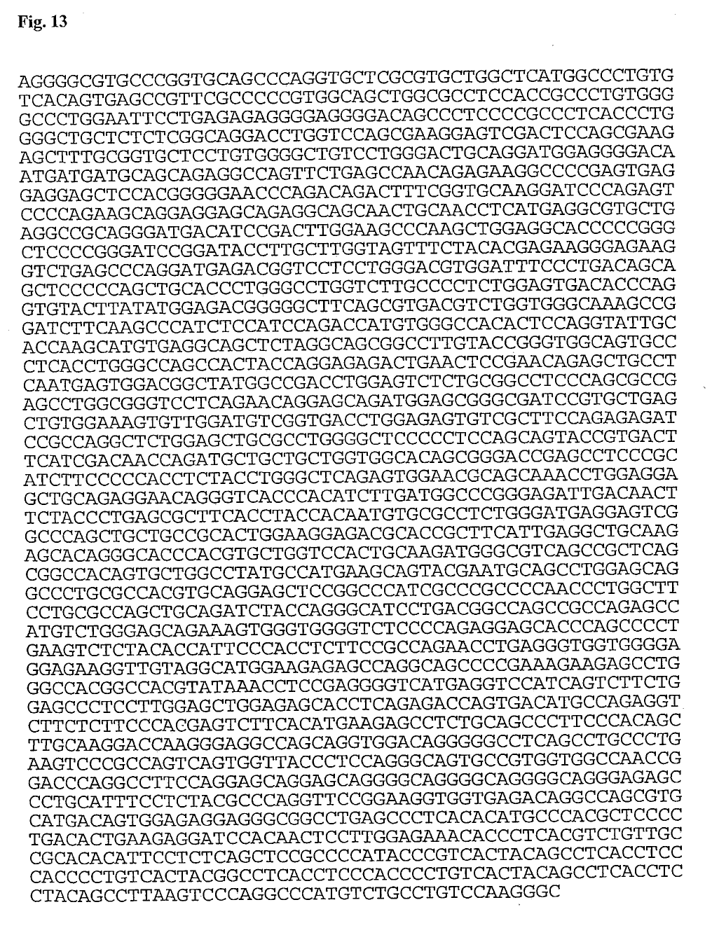

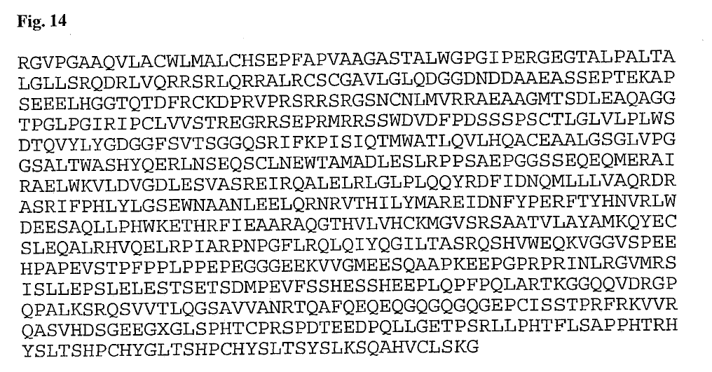

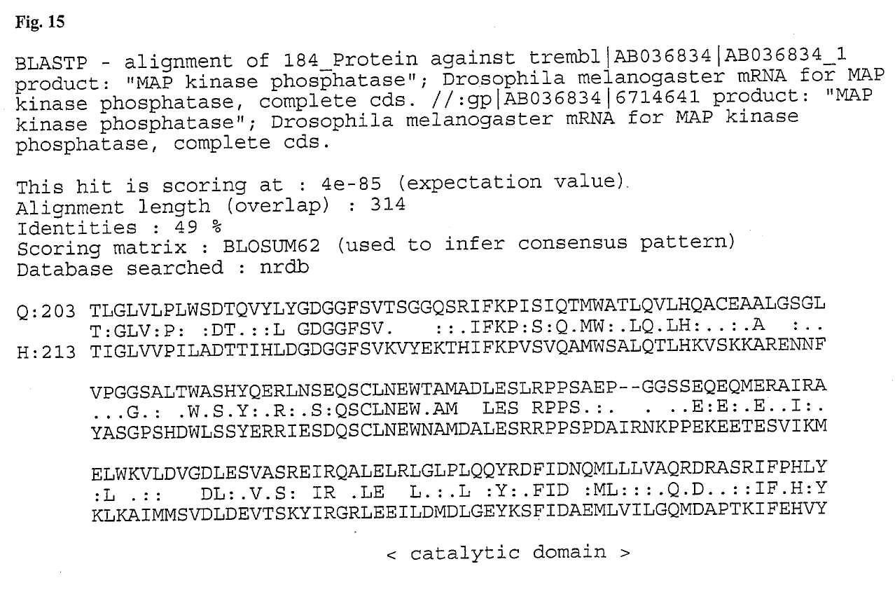

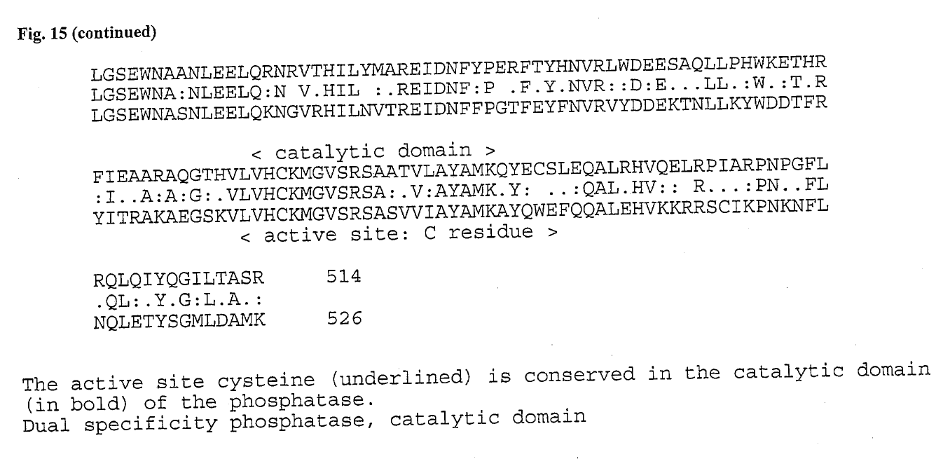

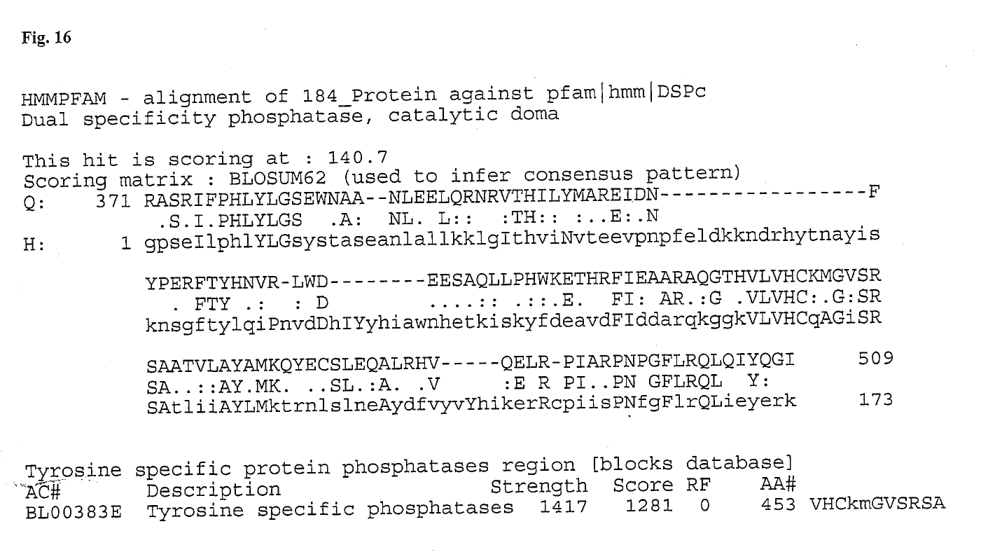

Human MAP kinase phosphatase-like enzyme is 54% identical over 109 amino acids to the Drosophila melanogaster protein identified with trembl Accession No. AB036834 and annotated as “MAP kinase phosphatase” (FIG. 10). Both pfam and BLOCKS searches confirm the protein's phosphatase function (FIGS. 11 and 12). In addition, the critical active site is found in the molecule (see FIG. 10). The coding sequence for human MAP kinase phosphatase-like enzyme (SEQ ID NO:1 or 10) is related to several ESTs (SEQ ID NOS:8-9), indicating that the coding sequence is expressed.

Human MAP kinase phosphatase-like enzyme is expected to be useful for the same purposes as previously identified MAP kinase phosphatase enzymes. Thus, human MAP kinase phosphatase-like enzyme can be used in therapeutic methods to treat disorders such as allergies including asthma, CNS disorders, diabetes, obesity, chronic obstructive pulmonary disease, and cardiovascular diseases. Human MAP kinase phosphatase-like enzyme also can be used to screen for human MAP kinase phosphatase-like enzyme agonists and antagonists.

Polypeptides

Human MAP kinase phosphatase-like enzyme polypeptides according to the invention comprise at least 6, 10, 15, 20, 25, 50, 75, 100, 125, or 140 contiguous amino acids selected from the amino acid sequence shown in SEQ ID NO:2 or 11 or a biologically active variant thereof, as defined below. A human MAP kinase phosphatase-like enzyme polypeptide of the invention therefore can be a portion of a human MAP kinase phosphatase-like enzyme, a full-length human MAP kinase phosphatase-like enzyme, or a fusion protein comprising all or a portion of a human MAP kinase phosphatase-like enzyme.

Biologically Active Variants

Human MAP kinase phosphatase-like enzyme polypeptide variants which are biologically active, e.g., retain the ability to hydrolyze protein tyrosine phosphate to tyrosine and orthophosphate, also are human MAP kinase phosphatase-like enzyme polypeptides. Preferably, naturally or non-naturally occurring MAP kinase phosphatase-like enzyme polypeptide variants have amino acid sequences which are at least about 50, 55, 60, 65, or 70, preferably about 75, 80, 85, 90, 96, 96, or 98% identical to the amino acid sequence shown in SEQ ID NO:2 or a fragment thereof. Percent identity between a putative polypeptide variant and an amino acid sequence of SEQ ID NO:2 or 11 is determined with the Needleman/Wunsch algorithm (Needleman and Wunsch, J.Mol. Biol. 48; 443-453, 1970) using a Blosum62 matrix with a gap creation penalty of 8 and a gap extension penalty of 2 (S. Henikoff and J. G. Henikoff, Proc. Natl. Acad. Sci. USA 89:10915-10919, 1992).

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of a MAP kinase phosphatase-like enzyme polypeptide can be found using computer programs well known in the art, such as DNASTAR software. Whether an amino acid change results in a biologically active polypeptide can readily be determined by assaying for MAP kinase phosphatase activity, as described, e.g., in the specific examples, below.

Fusion Proteins

Fusion proteins are useful for generating antibodies against MAP kinase phosphatase-like enzyme amino acid sequences and for use in various assay systems. For example, fusion proteins can be used to identify proteins which interact with portions of a MAP kinase phosphatase-like enzyme polypeptide. Protein affinity chromatography or library-based assays for protein-protein interactions, such as the yeast two-hybrid or phage display systems, can be used for this purpose. Such methods are well known in the art and also can be used as drug screens.

A MAP kinase phosphatase-like enzyme fusion protein comprises two polypeptide segments fused together by means of a peptide bond. The first polypeptide segment comprises at least 6, 10, 15, 20, 25, 50, 75, 100, 125, or 140 contiguous amino acids of SEQ ID NO:2 or 11 or of a biologically active variant, such as those described above. The first polypeptide segment also can comprise full-length MAP kinase phosphatase-like enzyme.

The second polypeptide segment can be a full-length protein or a protein fragment. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the MAP kinase phosphatase-like enzyme polypeptide-encoding sequence and the heterologous protein sequence, so that the desired polypeptide can be cleaved and purified away from the heterologous moiety.

A fusion protein can be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises coding sequences selected from the complement of SEQ ID NO:1 or 10 in proper reading frame with nucleotides encoding the second polypeptide segment and expressing the DNA construct in a host cell, as is known in the art. Many kits for constructing fusion proteins are available from companies such as Promega Corporation (Madison, Wis.), Stratagene (La Jolla, Calif.), CLONTECH (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Identification of Species Homologs

Species homologs of human MAP kinase phosphatase-like enzyme polypeptide can be obtained using MAP kinase phosphatase-like enzyme polypeptide polynucleotides (described below) to make suitable probes or primers for screening cDNA expression libraries from other species, such as mice, monkeys, or yeast, identifying cDNAs which encode homologs of MAP kinase phosphatase-like enzyme polypeptide, and expressing the cDNAs as is known in the art.

Polynucleotides

A MAP kinase phosphatase-like enzyme polynucleotide can be single- or double-stranded and comprises a coding sequence or the complement of a coding sequence for a MAP kinase phosphatase-like enzyme polypeptide. A coding sequence for MAP kinase phosphatase-like enzyme shown in SEQ ID NO:2 is shown in SEQ ID NO:1. This coding sequence is found within the larger genomic sequence shown in SEQ ID NO:4. A coding sequence for MAP kinase phosphatase-like enzyme shown in SEQ ID NO:10 is shown in SEQ ID NO:11.

Degenerate nucleotide sequences encoding human MAP kinase phosphatase-like enzyme polypeptides, as well as homologous nucleotide sequences which are at least about 50, 55, 60, 65, 70, preferably about 75, 90, 96, or 98% identical to the nucleotide sequence shown in SEQ ID NO:1 or 10 or its complement also are MAP kinase phosphatase-like enzyme polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of MAP kinase phosphatase-like enzyme polynucleotides which encode biologically active MAP kinase phosphatase-like enzyme polypeptides also are MAP kinase phosphatase-like enzyme poly-nucleotides.

Identification of Polynucleotide Variants and Homologs

Variants and homologs of the polynucleotides described above also are MAP kinase phosphatase-like enzyme polynucleotides. Typically, homologous polynucleotide sequences can be identified by hybridization of candidate polynucleotides to known MAP kinase phosphatase-like enzyme polynucleotides under stringent conditions, as is known in the art. For example, using the following wash conditions—2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each--homologous sequences can be identified which contain at most about 25-30% basepair mismatches. More preferably, homologous nucleic acid strands contain 15-25% basepair mismatches, even more preferably 5-15% basepair mismatches.

Species homologs of the MAP kinase phosphatase-like enzyme polynucleotides disclosed herein also can be identified by making suitable probes or primers and screening cDNA expression libraries from other species, such as mice, monkeys, or yeast. Human variants of MAP kinase phosphatase-like enzyme polynucleotides can be identified, for example, by screening human cDNA expression libraries. It is well known that the Tₘ of a double-stranded DNA decreases by 1-1.5° C. with every 1% decrease in homology (Bonner et al., J. Mol. Biol. 81, 123 (1973). Variants of human MAP kinase phosphatase-like enzyme polynucleotides or MAP kinase phosphatase-like enzyme polynucleotides of other species can therefore be identified by hybridizing a putative homologous polynucleotide with a polynucleotide having a nucleotide sequence of SEQ ID NO:1 or 10 or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to MAP kinase phosphatase-like enzyme polynucleotides or their complements following stringent hybridization and/or wash conditions also are MAP kinase phosphatase-like enzyme polynucleotides. Stringent wash conditions are well known and understood in the art and are disclosed, for example, in Sambrook et al., MOLECULAR CLONING: A LABORATORY MANUAL, 2d ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between a polynucleotide having a nucleotide sequence shown in SEQ ID NO:1 or 10 or the complement thereof and a polynucleotide sequence which is at least about 50, 55, 60, 65, 70, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tₘ=81.5° C.−16.6(log₁₀[Na⁺])+0.41(% G+C)−0.63(% formamide)−600/l),

where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

Preparation of Polynucleotides

A MAP kinase phosphatase-like enzyme polynucleotide can be isolated free of other cellular components such as membrane components, proteins, and lipids. Polynucleotides can be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art. Any such technique for obtaining a polynucleotide can be used to obtain isolated MAP kinase phosphatase-like enzyme polynucleotides. For example, restriction enzymes and probes can be used to isolate polynucleotide fragments which comprises MAP kinase phosphatase-like nucleotide sequences. Isolated polynucleotides are in preparations which are free or at least 70, 80, or 90% free of other molecules.

Human MAP kinase phosphatase-like enzyme cDNA molecules can be made with standard molecular biology techniques, using human MAP kinase phosphatase-like enzyme mRNA as a template. Human MAP kinase phosphatase-like enzyme cDNA molecules can thereafter be replicated using molecular biology techniques known in the art and disclosed in manuals such as Sambrook et al. (1989). An amplification technique, such as PCR, can be used to obtain additional copies of polynucleotides of the invention, using either human genomic DNA or cDNA as a template.

Alternatively, synthetic chemistry techniques can be used to synthesizes MAP kinase phosphatase-like enzyme polynucleotides. The degeneracy of the genetic code allows alternate nucleotide sequences to be synthesized which will encode a polypeptide having, for example, an amino acid sequence shown in SEQ ID NO:2 or 11 or a biologically active variant thereof.

Extending Polynucleotides

Various PCR-based methods can be used to extend the nucleic acid sequences disclosed herein to detect upstream sequences such as promoters and regulatory elements. For example, restriction-site PCR uses universal primers to retrieve unknown sequence adjacent to a known locus (Sarkar, PCR Methods Applic. 2, 318-322, 1993). Genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR also can be used to amplify or extend sequences using divergent primers based on a known region (Triglia et al., Nucleic Acids Res. 16, 8186, 1988). Primers can be designed using commercially available software, such as OLIGO 4.06 Primer Analysis software (National Biosciences Inc., Plymouth, Minn.), to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method which can be used is capture PCR, which involves PCR amplification of DNA fragments adjacent to a known sequence in human and yeast artificial chromosome DNA (Lagerstrom et al., PCR Methods Applic. 1, 111-119, 1991). In this method, multiple restriction enzyme digestions and ligations also can be used to place an engineered double-stranded sequence into an unknown fragment of the DNA molecule before performing PCR.

Another method which can be used to retrieve unknown sequences is that of Parker et al., Nucleic Acids Res. 19, 3055-3060, 1991). Additionally, PCR, nested primers, and PROMOTERFINDER libraries (CLONTECH, Palo Alto, Calif.) can be used to walk genomic DNA (CLONTECH, Palo Alto, Calif.). This process avoids the need to screen libraries and is useful in finding intron/exon junctions.

When screening for full-length cDNAs, it is preferable to use libraries that have been size-selected to include larger cDNAs. Randomly-primed libraries are preferable, in that they will contain more sequences which contain the 5′ regions of genes. Use of a randomly primed library may be especially preferable for situations in which an oligo d(T) library does not yield a full-length cDNA. Genomic libraries can be useful for extension of sequence into 5′ non-transcribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) which are laser activated, and detection of the emitted wavelengths by a charge coupled device camera. Output/light intensity can be converted to electrical signal using appropriate software (e.g. GENOTYPER and Sequence NAVIGATOR, Perkin Elmer), and the entire process from loading of samples to computer analysis and electronic data display can be computer controlled. Capillary electrophoresis is especially preferable for the sequencing of small pieces of DNA which might be present in limited amounts in a particular sample.

Obtaining Polypeptides

Human MAP kinase phosphatase-like enzyme polypeptides can be obtained, for example, by purification from human cells, by expression of MAP kinase phosphatase-like enzyme polynucleotides, or by direct chemical synthesis.

Protein Purification

Human MAP kinase phosphatase-like enzyme polypeptides can be purified from any cell which expresses the enzyme, including host cells which have been transfected with MAP kinase phosphatase-like enzyme expression constructs. Placenta (choriocarcinoma), kidney (renal cell adenocarcinoma), and colon tumor provide especially useful sources of MAP kinase phosphatase-like enzyme polypeptides. A purified MAP kinase phosphatase-like enzyme polypeptide is separated from other compounds which normally associate with the MAP kinase phosphatase-like enzyme polypeptide in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis. A preparation of purified MAP kinase phosphatase-like enzyme polypeptides is at least 80% pure; preferably, the preparations are 90%, 95%, or 99% pure. Purity of the preparations can be assessed by any means known in the art, such as SDS-polyacrylamide gel electrophoresis.

Expression of Polynucleotides

To express a human MAP kinase phosphatase-like enzyme polynucleotide, the polynucleotide can be inserted into an expression vector which contains the necessary elements for the transcription and translation of the inserted coding sequence. Methods which are well known to those skilled in the art can be used to construct expression vectors containing sequences encoding MAP kinase phosphatase-like enzyme polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., CURRENT PROTOCOLS IN MOLECULAR BIOLOGY, John Wiley & Sons, New York, N.Y., 1989.

A variety of expression vector/host systems can be utilized to contain and express sequences encoding a MAP kinase phosphatase-like enzyme polypeptide. These include, but are not limited to, microorganisms, such as bacteria transformed with recombinant bacteriophage, plasmid, or cosmid DNA expression vectors; yeast transformed with yeast expression vectors, insect cell systems infected with virus expression vectors (e.g., baculovirus), plant cell systems transformed with virus expression vectors (e.g., cauliflower mosaic virus, CaMV; tobacco mosaic virus, TMV) or with bacterial expression vectors (e.g., Ti or pBR322 plasmids), or animal cell systems.

The control elements or regulatory sequences are those non-translated regions of the vector—enhancers, promoters, 5′ and 3′ untranslated regions—which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding a MAP kinase phosphatase-like enzyme polypeptide, vectors based on SV40 or EBV can be used with an appropriate selectable marker.

Bacterial and Yeast Expression Systems

In bacterial systems, a number of expression vectors can be selected depending upon the use intended for the MAP kinase phosphatase-like enzyme polypeptide. For example, when a large quantity of a polypeptide is needed for the induction of antibodies, vectors which direct high level expression of fusion proteins that are readily purified can be used. Such vectors include, but are not limited to, multifunctional E. coli cloning and expression vectors such as BLUESCRIPT (Stratagene). In a BLUESCRIPT vector, a sequence encoding the polypeptide can be ligated into the vector in frame with sequences for the amino-terminal Met and the subsequent 7 residues of β-galactosidase so that a hybrid protein is produced. pIN vectors (Van Heeke & Schuster, J. Biol. Chem. 264, 5503-5509, 1989) or pGEX vectors (Promega, Madison, Wis.) also can be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems can be designed to include heparin, thrombin, or factor Xa protease cleavage sites so that the cloned polypeptide of interest can be released from the GST moiety at will. In the yeast Saccharomyces cerevisiae, a number of vectors containing constitutive or inducible promoters such as alpha factor, alcohol oxidase, and PGH can be used. For reviews, see Ausubel et al. (1989) and Grant et al., Methods Enzymol. 153, 516-544, 1987.

Plant and Insect Expression Systems

If plant expression vectors are used, the expression of sequences encoding MAP kinase phosphatase-like enzyme polypeptides can be driven by any of a number of promoters. For example, viral promoters such as the 35S and 19S promoters of CaMV can be used alone or in combination with the omega leader sequence from TMV (Takamatsu, EMBO J. 6, 307-311, 1987). Alternatively, plant promoters such as the small subunit of RUBISCO or heat shock promoters can be used (Coruzzi et al., EMBO J. 3, 1671-1680, 1984; Broglie et al., Science 224, 838-843, 1984; Winter et al., Results Probl. Cell Differ. 17, 85-105, 1991). These constructs can be introduced into plant cells by direct DNA transformation or by pathogen-mediated transfection. Such techniques are described in a number of generally available reviews (e.g., Hobbs or Murray, in MCGRAW HILL YEARBOOK OF SCIENCE AND TECHNOLOGY, McGraw Hill, New York, N.Y., pp. 191-196, 1992).

An insect system also can be used to express a MAP kinase phosphatase-like enzyme polypeptide. For example, in one such system Autographa californica nuclear polyhedrosis virus (AcNPV) is used as a vector to express foreign genes in Spodoptera frugiperda cells or in Trichoplusia larvae. Sequences encoding MAP kinase phosphatase-like enzyme polypeptides can be cloned into a non-essential region of the virus, such as the polyhedrin gene, and placed under control of the polyhedrin promoter. Successful insertion of MAP kinase phosphatase-like enzyme polypeptides will render the polyhedrin gene inactive and produce recombinant virus lacking coat protein. The recombinant viruses can then be used to infect S. frugiperda cells or Trichoplusia larvae in which MAP kinase phosphatase-like enzyme polypeptides can be expressed (Engelhard et al., Proc. Nat. Acad. Sci. 91, 3224-3227, 1994).

Mammalian Expression Systems

A number of viral-based expression systems can be used to express MAP kinase phosphatase-like enzyme polypeptides in mammalian host cells. For example, if an adenovirus is used as an expression vector, sequences encoding MAP kinase phosphatase-like enzyme polypeptides can be ligated into an adenovirus transcription/translation complex comprising the late promoter and tripartite leader sequence. Insertion in a non-essential E1 or E3 region of the viral genome can be used to obtain a viable virus which is capable of expressimg a MAP kinase phosphatase-like enzyme polypeptide in infected host cells (Logan & Shenk, Proc. Natl. Acad. Sci. 81, 3655-3659, 1984). If desired, transcription enhancers, such as the Rous sarcoma virus (RSV) enhancer, can be used to increase expression in mammalian host cells.

Human artificial chromosomes (HACs) also can be used to deliver larger fragments of DNA than can be contained and expressed in a plasmid. HACs of 6M to 10M are constructed and delivered to cells via conventional delivery methods (e.g., liposomes, polycationic amino polymers, or vesicles).

Specific initiation signals also can be used to achieve more efficient translation of sequences encoding MAP kinase phosphatase-like enzyme polypeptides. Such signals include the ATG initiation codon and adjacent sequences. In cases where sequences encoding a MAP kinase phosphatase-like enzyme polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals (including the ATG initiation codon) should be provided. The initiation codon should be in the correct reading frame to ensure translation of the entire insert. Exogenous translational elements and initiation codons can be of various origins, both natural and synthetic. The efficiency of expression can be enhanced by the inclusion of enhancers which are appropriate for the particular cell system which is used (see Scharf et al., Results Probl. Cell Differ. 20, 125-162, 1994).

Host Cells

A host cell strain can be chosen for its ability to modulate the expression of the inserted sequences or to process the expressed MAP kinase phosphatase-like enzyme polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding and/or function. Different host cells which have specific cellular machinery and characteristic mechanisms for post-translational activities (e.g., CHO, HeLa, MDCK, HEK293, and WI38), are available from the American Type Culture Collection (ATCC; 10801 University Boulevard, Manassas, Va. 20110-2209) and can be chosen to ensure the correct modification and processing of the foreign protein.

Stable expression is preferred for long-term, high-yield production of recombinant proteins. For example, cell lines which stably express MAP kinase phosphatase-like enzyme polypeptides can be transformed using expression vectors which can contain viral origins of replication and/or endogenous expression elements and a selectable marker gene on the same or on a separate vector. Following the introduction of the vector, cells can be allowed to grow for 1-2 days in an enriched medium before they are switched to a selective medium. The purpose of the selectable marker is to confer resistance to selection, and its presence allows growth and recovery of cells which successfully express the introduced MAP kinase phosphatase-like enzyme sequences. Resistant clones of stably transformed cells can be proliferated using tissue culture techniques appropriate to the cell type. See, for example, ANIMAL CELL CULTURE, R. I. Freshney, ed., 1986.

Any number of selection systems can be used to recover transformed cell lines. These include, but are not limited to, the herpes simplex virus thymidine kinase (Wigler et al., Cell 11, 223-32, 1977) and adenine phosphoribosyltransferase (Lowy et al., Cell 22, 817-23, 1980) genes which can be employed in tk⁻ or aprt⁻ cells, respectively. Also, antimetabolite, antibiotic, or herbicide resistance can be used as the basis for selection. For example, dhfr confers resistance to methotrexate (Wigler et al., Proc. Natl. Acad. Sci. 77, 3567-70, 1980), npt confers resistance to the aminoglycosides, neomycin and G-418 (Colbere-Garapin et al., J. Mol. Biol. 150, 1-14, 1981), and als and pat confer resistance to chlorsulfuron and phosphinotricin acetyltransferase, respectively (Murray, 1992, supra). Additional selectable genes have been described. For example, trpB allows cells to utilize indole in place of tryptophan, or hisD, which allows cells to utilize histinol in place of histidine (Hartman & Mulligan, Proc. Natl. Acad. Sci. 85, 8047-51, 1988). Visible markers such as anthocyanins, β-glucuronidase and its substrate GUS, and luciferase and its substrate luciferin, can be used to identify transformants and to quantify the amount of transient or stable protein expression attributable to a specific vector system (Rhodes et al., Methods Mol. Biol. 55, 121-131, 1995).

Detecting Expression

Although the presence of marker gene expression suggests that the MAP kinase phosphatase-like enzyme polynucleotide is also present, its presence and expression may need to be confirmed. For example, if a sequence encoding a MAP kinase phosphatase-like enzyme polypeptide is inserted within a marker gene sequence, transformed cells containing sequences which encode a MAP kinase phosphatase-like enzyme polypeptide can be identified by the absence of marker gene function. Alternatively, a marker gene can be placed in tandem with a sequence encoding a MAP kinase phosphatase-like enzyme polypeptide under the control of a single promoter. Expression of the marker gene in response to induction or selection usually indicates expression of the MAP kinase phosphatase-like enzyme polynucleotide.

Alternatively, host cells which contain a MAP kinase phosphatase-like enzyme polynucleotide and which express a MAP kinase phosphatase-like enzyme polypeptide can be identified by a variety of procedures known to those of skill in the art. These procedures include, but are not limited to, DNA-DNA or DNA-RNA hybridizations and protein bioassay or immunoassay techniques which include membrane, solution, or chip-based technologies for the detection and/or quantification of nucleic acid or protein. For example, the presence of a polynucleotide sequence encoding a MAP kinase phosphatase-like enzyme polypeptide can be detected by DNA-DNA or DNA-RNA hybridization or amplification using probes or fragments or fragments of polynucleotides encoding a MAP kinase phosphatase-like enzyme polypeptide. Nucleic acid amplification-based assays involve the use of oligonucleotides selected from sequences encoding a MAP kinase phosphatase-like enzyme polypeptide to detect transformants which contain a MAP kinase phosphatase-like enzyme polynucleotide.

A variety of protocols for detecting and measuring the expression of a MAP kinase phosphatase-like enzyme polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a MAP kinase phosphatase-like enzyme polypeptide can be used, or a competitive binding assay can be employed. These and other assays are described in Hampton et al., SEROLOGICAL METHODS: A LABORATORY MANUAL, APS Press, St. Paul, Minn., 1990) and Maddox et al., J. Exp. Med. 158, 1211-1216, 1983).

A wide variety of labels and conjugation techniques are known by those skilled in the art and can be used in various nucleic acid and amino acid assays. Means for producing labeled hybridization or PCR probes for detecting sequences related to polynucleotides encoding MAP kinase phosphatase-like enzyme polypeptides include oligolabeling, nick translation, end-labeling, or PCR amplification using a labeled nucleotide. Alternatively, sequences encoding a MAP kinase phosphatase-like enzyme polypeptide can be cloned into a vector for the production of an mRNA probe. Such vectors are known in the art, are commercially available, and can be used to synthesize RNA probes in vitro by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or cloromogenic agents, as well as substrates, cofactors, inhibitors, magnetic particles, and the like.

Expression and Purification of Polypeptides

Host cells transformed with nucleotide sequences encoding a MAP kinase phosphatase-like enzyme polypeptide can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing polynucleotides which encode MAP kinase phosphatase-like enzyme polypeptides can be designed to contain signal sequences which direct secretion of soluble MAP kinase phosphatase-like enzyme polypeptides through a prokaryotic or eukaryotic cell membrane or which direct the membrane insertion of membrane-bound MAP kinase phosphatase-like enzyme polypeptide.

As discussed above, other constructions can be used to join a sequence encoding a MAP kinase phosphatase-like enzyme polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Inclusion of cleavable linker sequences such as those specific for Factor Xa or enterokinase (Invitrogen, San Diego, Calif.) between the purification domain and the MAP kinase phosphatase-like enzyme polypeptide also can be used to facilitate purification. One such expression vector provides for expression of a fusion protein containing a MAP kinase phosphatase-like enzyme polypeptide and 6 histidine residues preceding a thioredoxin or an enterokinase cleavage site. The histidine residues facilitate purification by IMAC (immobilized metal ion affinity chromatography, as described in Porath et al., Prot. Exp. Purif. 3, 263-281, 1992), while the enterokinase cleavage site provides a means for purifying the MAP kinase phosphatase-like enzyme polypeptide from the fusion protein. Vectors which contain fusion proteins are disclosed in Kroll et al., DNA Cell Biol. 12, 441-453, 1993.

Chemical Synthesis

Sequences encoding a MAP kinase phosphatase-like enzyme polypeptide can be synthesized, in whole or in part, using chemical methods well known in the art (see Caruthers et al., Nucl. Acids Res. Symp. Ser. 215-223, 1980; Horn et al. Nucl. Acids Res. Symp. Ser. 225-232, 1980). Alternatively, a MAP kinase phosphatase-like enzyme polypeptide itself can be produced using chemical methods to synthesize its amino acid sequence, such as by direct peptide synthesis using solid-phase techniques (Merrifield, J. Am. Chem. Soc. 85, 2149-2154, 1963; Roberge et al., Science 269, 202-204, 1995). Protein synthesis can be performed using manual techniques or by automation. Automated synthesis can be achieved, for example, using Applied Biosystems 431A Peptide Synthesizer (Perkin Elmer). Optionally, fragments of MAP kinase phosphatase-like enzyme polypeptides can be separately synthesized and combined using chemical methods to produce a full-length molecule.

The newly synthesized peptide can be substantially purified by preparative high performance liquid chromatography (e.g., Creighton, PROTEINS: STRUCTURES AND MOLECULAR PRINCIPLES, W H Freeman and Co., New York, N.Y., 1983). The composition of a synthetic MAP kinase phosphatase-like enzyme polypeptide can be confirmed by amino acid analysis or sequencing (e.g., the Edman degradation procedure; see Creighton, supra). Additionally, any portion of the amino acid sequence of the MAP kinase phosphatase-like enzyme polypeptide can be altered during direct synthesis and/or combined using chemical methods with sequences from other proteins to produce a variant polypeptide or a fusion protein.

Production of Altered Polypeptides

As will be understood by those of skill in the art, it may be advantageous to produce MAP kinase phosphatase-like enzyme polypeptide-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life which is longer than that of a transcript generated from the naturally occurring sequence.

The nucleotide sequences disclosed herein can be engineered using methods generally known in the art to alter MAP kinase phosphatase-like enzyme polypeptide-encoding sequences for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

Antibodies

Any type of antibody known in the art can be generated to bind specifically to an epitope of a MAP kinase phosphatase-like enzyme polypeptide. “Antibody” as used herein includes intact immunoglobulin molecules, as well as fragments thereof, such as Fab, F(ab′)₂, and Fv, which are capable of binding an epitope of a MAP kinase phosphatase-like enzyme polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of a MAP kinase phosphatase-like enzyme polypeptide can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

Typically, an antibody which specifically binds to a MAP kinase phosphatase-like enzyme polypeptide provides a detection signal at least 5-, 10-, or 20-fold higher than a detection signal provided with other proteins when used in an immunochemical assay. Preferably, antibodies which specifically bind to MAP kinase phosphatase-like enzyme polypeptides do not detect other proteins in immunochemical assays and can immunoprecipitate a MAP kinase phosphatase-like enzyme polypeptide from solution.

Human MAP kinase phosphatase-like enzyme polypeptides can be used to immunize a mammal, such as a mouse, rat, rabbit, guinea pig, monkey, or human, to produce polyclonal antibodies. If desired, a MAP kinase phosphatase-like enzyme polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacteriun parvum are especially useful.

Monoclonal antibodies which specifically bind to a MAP kinase phosphatase-like enzyme polypeptide can be prepared using any technique which provides for the production of antibody molecules by continuous cell lines in culture. These techniques include, but are not limited to, the hybridoma technique, the human B-cell hybridoma technique, and the EBV-hybridoma technique (Kohler et al., Nature 256, 495-497, 1985; Kozbor et al., J. Immunol. Methods 81, 31-42, 1985; Cote et al., Proc. Natl. Acad Sci. 80, 2026-2030, 1983; Cole et al., Mol. Cell Biol. 62, 109-120, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-6855, 1984; Neuberger et al., Nature 312, 604-608, 1984; Takeda et al., Nature 314, 452-454, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions. Alternatively, humanized antibodies can be produced using recombinant methods, as described in GB2188638B. Antibodies which specifically bind to a MAP kinase phosphatase-like enzyme polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to MAP kinase phosphatase-like enzyme polypeptides. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., 1996, Eur. J. Cancer Prev. 5, 507-11). Single-chain antibodies can be mono- or bispecific, and can be bivalent or tetravalent. Construction of tetravalent, bispecific single-chain antibodies is taught, for example, in Coloma & Morrison, 1997, Nat. Biotechnol. 15, 159-63. Construction of bivalent, bispecific single-chain antibodies is taught in Mallender & Voss, 1994, J. Biol. Chem. 269, 199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., 1995, Int. J. Cancer 61, 497-501; Nicholls et al., 1993, J. Immunol. Meth. 165, 81-91).

Antibodies which specifically bind to MAP kinase phosphatase-like enzyme polypeptides also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833-3837, 1989; Winter et al., Nature 349, 293-299, 1991).

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies according to the invention can be purified by methods well known in the art. For example, antibodies can be affinity purified by passage over a column to which a MAP kinase phosphatase-like enzyme polypeptide is bound. The bound antibodies can then be eluted from the column using a buffer with a high salt concentration.

Antisense Oligonucleotides

Antisense oligonucleotides are nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of MAP kinase phosphatase-like enzyme gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, Meth. Mol. Biol. 20, 1-8, 1994; Sonveaux, Meth. Mol. Biol. 26, 1-72, 1994; Uhlmann et al., Chem. Rev. 90, 543-583, 1990.

Modifications of MAP kinase phosphatase-like enzyme gene expression can be obtained by designing antisense oligonucleotides which will form duplexes to the control, 5′, or regulatory regions of the MAP kinase phosphatase-like enzyme gene. Oligonucleotides derived from the transcription initiation site, e.g., between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, MOLECULAR AND IMMUNOLOGIC APPROACHES, Futura Publishing Co., Mt. Kisco, N.Y., 1994). An antisense oligonucleotide also can be designed to block translation of mRNA by preventing the transcript from binding to ribosomes.

Precise complementarity is not required for successful complex formation between an antisense oligonucleotide and the complementary sequence of a MAP kinase phosphatase-like enzyme polynucleotide. Antisense oligonucleotides which comprise, for example, 2, 3, 4, or 5 or more stretches of contiguous nucleotides which are precisely complementary to a MAP kinase phosphatase-like enzyme polynucleotide, each separated by a stretch of contiguous nucleotides which are not complementary to adjacent MAP kinase phosphatase-like enzyme nucleotides, can provide sufficient targeting specificity for MAP kinase phosphatase-like enzyme mRNA. Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular MAP kinase phosphatase-like enzyme polynucleotide sequence.

Antisense oligonucleotides can be modified without affecting their ability to hybridize to a MAP kinase phosphatase-like enzyme polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′-substituted oligonucleotide in which the 3′ hydroxyl group or the 5′ phosphate group are substituted, also can be employed in a modified antisense oligonucleotide. These modified oligonucleotides can be prepared by methods well known in the art. See, e.g., Agrawal et al., Trends Biotechnol. 10, 152-158, 1992; Uhlmann et al., Chem. Rev. 90, 543-584, 1990; Uhlmann et al., Tetrahedron. Lett. 215, 3539-3542, 1987.

Ribozymes

Ribozymes are RNA molecules with catalytic activity. See, e.g., Cech, Science 236, 1532-1539; 1987; Cech, Ann. Rev. Biochem. 59, 543-568; 1990, Cech, Curr. Opin. Struct. Biol. 2, 605-609; 1992, Couture & Stinchcomb, Trends Genet. 12, 510-515, 1996. Ribozymes can be used to inhibit gene function by cleaving an RNA sequence, as is known in the art (e.g., Haseloff et al., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of a MAP kinase phosphatase-like enzyme polynucleotide can be used to generate ribozymes which will specifically bind to mRNA transcribed from the MAP kinase phosphatase-like enzyme polynucleotide. Methods of designing and constructing ribozymes which can cleave other RNA molecules in trans in a highly sequence specific manner have been developed and described in the art (see Haseloff et al. Nature 334, 585-591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach et al., EP 321,201).

Specific ribozyme cleavage sites within a MAP kinase phosphatase-like enzyme RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate MAP kinase phosphatase-like enzyme RNA targets also can be evaluated by testing accessibility to hybridization with complementary oligonucleotides using ribonuclease protection assays. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease MAP kinase phosphatase-like enzyme expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells.

As taught in Haseloff et al., U.S. Pat. No. 5,641,673, ribozymes can be engineered so that ribozyme expression will occur in response to factors which induce expression of a target gene. Ribozymes also can be engineered to provide an additional level of regulation, so that destruction of mRNA occurs only when both a ribozyme and a target gene are induced in the cells.

Identification of Target and Pathway Genes and Proteins

Described herein are methods for the identification of genes whose products interact with human MAP kinase phosphatase-like enzyme. Such genes may represent genes which are differentially expressed in disorders including, but not limited to, allergies including asthma, CNS disorders, diabetes, obesity, chronic obstructive pulmonary disease, cancer, and cardiovascular diseases. Further, such genes may represent genes which are differentially regulated in response to manipulations relevant to the progression or treatment of such diseases. Such differentially expressed genes may represent “target” and/or “fingerprint” genes. Methods for the identification of such differentially expressed genes are described below. Methods for the further characterization of such differentially expressed genes, and for their identification as target and/or fingerprint genes also are described below.

In addition, methods are described for the identification of genes, termed “pathway genes,” which are involved in a disorder of interest. “Pathway gene,” as used herein, refers to a gene whose gene product exhibits the ability to interact with gene products involved in these disorders. A pathway gene may be differentially expressed and, therefore, may have the characteristics of a target and/or fingerprint gene.

“Differential expression” refers to both quantitative as well as qualitative differences in a gene's temporal and/or tissue expression pattern. Thus, a differentially expressed gene may qualitatively have its expression activated or completely inactivated in normal versus diseased states, or under control versus experimental conditions. Such a qualitatively regulated gene will exhibit an expression pattern within a given tissue or cell type which is detectable in either normal or diseased subjects, but is not detectable in both. Alternatively, such a qualitatively regulated gene will exhibit an expression pattern within a given tissue or cell type which is detectable in either control or experimental subjects, but is not detectable in both. “Detectable” refers to an RNA expression pattern which is detectable via the standard techniques of differential display, RT-PCR and/or Northern analyses, which are well known to those of skill in the art.

A differentially expressed gene may have its expression modulated, i.e., quantitatively increased or decreased, in normal versus diseased states, or under control versus experimental conditions. The degree to which expression differs in a normal versus a diseased state need only be large enough to be visualized via standard characterization techniques, such as, for example, the differential display technique described below. Other such standard characterization techniques by which expression differences may be visualized include but are not limited to, quantitative RT (reverse transcriptase) PCR and Northern analyses.

Differentially expressed genes may be further described as target genes and/or fingerprint genes. “Fingerprint gene” refers to a differentially expressed gene whose expression pattern may be utilized as part of a prognostic or diagnostic evaluation, or which, alternatively, may be used in methods for identifying compounds useful for the treatment of various disorders. A fingerprint gene may also have the characteristics of a target gene or a pathway gene.

“Target gene” refers to a differentially expressed gene involved in a disorder of interest by which modulation of the level of target gene expression or of target gene product activity may act to ameliorate symptoms. A target gene may also have the characteristics of a fingerprint gene and/or a pathway gene.

Identification of Differentially Expressed Genes

A variety of methods may be utilized for the identification of genes which are involved in a disorder of interest. To identify differentially expressed genes, RNA, either total or mRNA, may be isolated from one or more tissues of the subjects utilized in paradigms such as those described above. RNA samples are obtained from tissues of experimental subjects and from corresponding tissues of control subjects. Any RNA isolation technique which does not select against the isolation of mRNA may be utilized for the purification of such RNA samples. See, for example, Ausubel et al., eds., CURRENT PROTOCOLS IN MOLECULAR BIOLOGY, John Wiley & Sons, Inc. New York, 1987-1993. Large numbers of tissue samples may readily be processed using techniques well known to those of skill in the art, such as, for example, the single-step RNA isolation process of Chomczynski, U.S. Pat. No. 4,843,155.

Transcripts within the collected RNA samples which represent RNA produced by differentially expressed genes may be identified by utilizing a variety of methods which are well known to those of skill in the art. For example, differential screening (Tedder et al., Proc. Natl. Acad. Sci. U.S.A. 85, 208-12, 1988), subtractive hybridization (Hedrick et al., Nature 308, 149-53; Lee et al., Proc. Natl. Acad. Sci. U.S.A. 88, 2825, 1984), and, preferably, differential display (Liang & Pardee, Science 257, 967-71, 1992; U.S. Pat. No. 5,262,311), may be utilized to identify nucleic acid sequences derived from genes that are differentially expressed.

Differential screening involves the duplicate screening of a cDNA library in which one copy of the library is screened with a total cell cDNA probe corresponding to the mRNA population of one cell type while a duplicate copy of the cDNA library is screened with a total cDNA probe corresponding to the mRNA population of a second cell type. For example, one cDNA probe may correspond to a total cell cDNA probe of a cell type or tissue derived from a control subject, while the second cDNA probe may correspond to a total cell cDNA probe of the same cell type or tissue derived from an experimental subject. Those clones which hybridize to one probe but not to the other potentially represent clones derived from genes differentially expressed in the cell type of interest in control versus experimental subjects.

Subtractive hybridization techniques generally involve the isolation of mRNA taken from two different sources, e.g., control and experimental tissue or cell type, the hybridization of the mRNA or single-stranded cDNA reverse-transcribed from the isolated mRNA, and the removal of all hybridized, and therefore double-stranded, sequences. The remaining non-hybridized, single-stranded cDNAs, potentially represent clones derived from genes that are differentially expressed in the two mRNA sources. Such single-stranded cDNAs are then used as the starting material for the construction of a library comprising clones derived from differentially expressed genes.

The differential display technique describes a procedure, utilizing the well known polymerase chain reaction (PCR; the experimental embodiment set forth in Mullis, U.S. Pat. No. 4,683,202), which allows for the identification of sequences derived from genes which are differentially expressed. First, isolated RNA is reverse-transcribed into single-stranded cDNA, utilizing standard techniques which are well known to those of skill in the art. Primers for the reverse transcriptase reaction may include, but are not limited to, oligo dT-containing primers.

Next, this technique uses pairs of PCR primers, as described below, which allow for the amplification of clones representing a random subset of the RNA transcripts present within any given cell. Utilizing different pairs of primers allows each of the mRNA transcripts present in a cell to be amplified. Among such amplified transcripts may be identified those which have been produced from differentially expressed genes.

The 3′ oligonucleotide primer of the primer pairs may contain an oligo dT stretch of 10-13, preferably 11, dT nucleotides at its 5′ end, which hybridizes to the poly(A) tail of mRNA or to the complement of a cDNA reverse transcribed from an mRNA poly(A) tail. Second, in order to increase the specificity of the 3′ primer, the primer may contain one or more, preferably two, additional nucleotides at its 3′ end. Because, statistically, only a subset of the mRNA derived sequences present in the sample of interest will hybridize to such primers, the additional nucleotides allow the primers to amplify only a subset of the mRNA derived sequences present in the sample of interest. This is preferred in that it allows more accurate and complete visualization and characterization of each of the bands representing amplified sequences.

The 5′ primer may contain a nucleotide sequence expected, statistically, to have the ability to hybridize to cDNA sequences derived from the tissues of interest. The nucleotide sequence may be an arbitrary one, and the length of the 5′ oligonucleotide primer may range from about 9 to about 15 nucleotides, with about 13 nucleotides being preferred. Arbitrary primer sequences cause the lengths of the amplified partial cDNAs produced to be variable, thus allowing different clones to be separated by using standard denaturing sequencing gel electrophoresis.

PCR reaction conditions should be chosen which optimize amplified product yield and specificity, and, additionally, produce amplified products of lengths which may be resolved utilizing standard gel electrophoresis techniques. Such reaction conditions are well known to those of skill in the art, and important reaction parameters include, for example, length and nucleotide sequence of oligonucleotide primers as discussed above, and annealing and elongation step temperatures and reaction times.

The pattern of clones resulting from the reverse transcription and amplification of the mRNA of two different cell types is displayed via sequencing gel electrophoresis and compared. Differentially expressed genes are indicated by differences in the two banding patterns.

Once potentially differentially expressed gene sequences have been identified via bulk techniques such as, for example, those described above, the differential expression of such putatively differentially expressed genes should be corroborated. Corroboration may be accomplished via, for example, such well known techniques as Northern analysis, quantitative RT PCR or RNase protection. Upon corroboration, the differentially expressed genes may be further characterized, and may be identified as target and/or fingerprint genes, as discussed below.

Amplified sequences of differentially expressed genes obtained through, for example, differential display may be used to isolate full length clones of the corresponding gene. The full length coding portion of the gene may readily be isolated, without undue experimentation, by molecular biological techniques well known in the art. For example, the isolated differentially expressed amplified fragment may be labeled and used to screen a cDNA library. Alternatively, the labeled fragment may be used to screen a genomic library.

PCR technology may also be utilized to isolate full length cDNA sequences. As described above, the isolated, amplified gene fragments obtained through differential display have 5′ terminal ends at some random point within the gene and usually have 3′ terminal ends at a position corresponding to the 3′ end of the transcribed portion of the gene. Once nucleotide sequence information from an amplified fragment is obtained, the remainder of the gene (i. e., the 5′ end of the gene, when utilizing differential display) may be obtained using, for example, RT-PCR.

In one embodiment of such a procedure for the identification and cloning of full length gene sequences, RNA may be isolated, following standard procedures, from an appropriate tissue or cellular source. A reverse transcription reaction may then be performed on the RNA using an oligonucleotide primer complimentary to the mRNA that corresponds to the amplified fragment, for the priming of first strand synthesis. Because the primer is anti-parallel to the mRNA, extension will proceed toward the 5′ end of the mRNA. The resulting RNA/DNA hybrid may then be “tailed” with guanines using a standard terminal transferase reaction, the hybrid may be digested with RNAase H, and second strand synthesis may then be primed with a poly-C primer. Using the two primers, the 5′ portion of the gene is amplified using PCR. Sequences obtained may then be isolated and recombined with previously isolated sequences to generate a full-length cDNA of the differentially expressed genes of the invention. For a review of cloning strategies and recombinant DNA techniques, see e.g., Sambrook et al., 1989, and Ausubel et al., 1989.

Identification of Pathway Genes

Methods are described herein for the identification of pathway genes. “Pathway gene” refers to a gene whose gene product exhibits the ability to interact with gene products involved in a disorder of interest. A pathway gene may be differentially expressed and, therefore, may have the characteristics of a target and/or fingerprint gene.

Any method suitable for detecting protein-protein interactions may be employed for identifying pathway gene products by identifying interactions between gene products and gene products known to be involved in a disorder of interest. Such known gene products may be cellular or extracellular proteins. Those gene products which interact with such known gene products represent pathway gene products and the genes which encode them represent pathway genes.

Among the traditional methods which may be employed are co-immunoprecipitation, crosslinking and co-purification through gradients or chromatographic columns. Utilizing procedures such as these allows for the identification of pathway gene products. Once identified, a pathway gene product may be used, in conjunction with standard techniques, to identify its corresponding pathway gene. For example, at least a portion of the amino acid sequence of the pathway gene product may be ascertained using techniques well known to those of skill in the art, such as via the Edman degradation technique (see, e.g., Creighton, PROTEINS: STRUCTURES AND MOLECULAR PRINCIPLES, W. H. Freeman & Co., N.Y., pp.34-49, 1983). The amino acid sequence obtained may be used as a guide for the generation of oligonucleotide mixtures that can be used to screen for pathway gene sequences. Screening made be accomplished, for example, by standard hybridization or PCR techniques. Techniques for the generation of oligonucleotide mixtures and the screening are well-known. (see, e.g., Ausubel, 1989, and Innis et al., eds., PCR PROTOCOLS: A GUIDE TO METHODS AND APPLICATIONS, 1990, Academic Press, Inc., New York).

Methods may be employed which result in the simultaneous identification of pathway genes which encode the protein interacting with a protein involved in a disorder of interest. These methods include, for example, probing expression libraries with labeled protein known or suggested to be involved in such disorders, using this protein in a manner similar to the well known technique of antibody probing of μgt11 libraries.

One method which detects protein interactions in vivo, the two-hybrid system, is described in detail for illustration only and not by way of limitation. One version of this system is been described in Chien et al., 1991, Proc. Natl. Acad. Sci. U.S.A. 88, 9578-82, 1991, and is commercially available from Clontech (Palo Alto, Calif.). Briefly, utilizing such a system, plasmids are constructed that encode two hybrid proteins: one consists of the DNA-binding domain of a transcription activator protein fused to a known protein, in this case, a protein known to be involved in a disorder of interest and the other consists of the transcription activator protein's activation domain fused to an unknown protein that is encoded by a cDNA which has been recombined into this plasmid as part of a cDNA library. The plasmids are transformed into a strain of the yeast Saccharomyces cerevisiae that contains a reporter gene (e.g., lacZ) whose regulatory region contains the transcription activator's binding sites. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with a known “bait” gene product. By way of example, and not by way of limitation, gene products known to be involved in a disorder of interest may be used as the bait gene products. These include but are not limited to the intracellular domain of receptors for such hormones as neuropeptide Y, galanin, interostatin, insulin, and CCK. Total genomic or cDNA sequences are fused to the DNA encoding an activation domain. This library and a plasmid encoding a hybrid of the bait gene product fused to the DNA-binding domain are cotransformed into a yeast reporter strain, and the resulting transformants are screened for those that express the reporter gene. For example, and not by way of limitation, the bait gene can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait gene product are to be detected can be made using methods routinely practiced in the art.

According to the particular system described herein, for example, the cDNA fragments can be inserted into a vector such that they are translationally fused to the activation domain of GAL4. This library can be co-transformed along with the bait gene-GAL4 fusion plasmid into a yeast strain which contains a lacZ gene driven by a promoter which contains GAL4 activation sequence. A cDNA encoded protein, fused to GAL4 activation domain, that interacts with bait gene product will reconstitute an active GAL4 protein and thereby drive expression of the lacZ gene. Colonies which express lacZ can be detected by their blue color in the presence of X-gal. The cDNA can then be purified from these strains, and used to produce and isolate the bait gene-interacting protein using techniques routinely practiced in the art. Once a pathway gene has been identified and isolated, it may be further characterized, as described below.

Characterization of Differentially Expressed and Pathway Genes

Differentially expressed and pathway genes, such as those identified via the methods discussed above, as well as genes identified by alternative means, may be further characterized by utilizing, for example, methods such as those discussed herein. Such genes will be referred to herein as “identified genes.” Analyses such as those described herein, yield information regarding the biological function of the identified genes. An assessment of the biological function of the differentially expressed genes, in addition, will allow for their designation as target and/or fingerprint genes.

Specifically, any of the differentially expressed genes whose further characterization indicates that a modulation of the gene's expression or a modulation of the gene product's activity may ameliorate any of the disorders of interest will be designated “target genes,” as defined above. Such target genes and target gene products, along with those discussed below, will constitute the focus of the compound discovery strategies discussed below. Further, such target genes, target gene products and/or modulating compounds can be used as part of the treatment methods described below.

Any of the differentially expressed genes whose further characterization indicates that such modulations may not positively affect a disorder of interest, but whose expression pattern contributes to a gene expression “fingerprint” pattern correlative of, for example, a malignant state will be designated a “fingerprint gene.” It should be noted that each of the target genes may also function as fingerprint genes, as well as may all or a portion of the pathway genes.

Pathway genes may also be characterized according to techniques such as those described herein. Those pathway genes which yield information indicating that they are differentially expressed and that modulation of the gene's expression or a modulation of the gene product's activity may ameliorate any of the disorders of interest will be also be designated “target genes.” Such target genes and target gene products, along with those discussed above, will constitute the focus of the compound discovery strategies discussed below and can be used as part of treatment methods.

Characterization of one or more of the pathway genes may reveal a lack of differential expression, but evidence that modulation of the gene's activity or expression may, nonetheless, ameliorate symptoms. In such cases, these genes and gene products would also be considered a focus of the compound discovery strategies. In instances wherein a pathway gene's characterization indicates that modulation of gene expression or gene product activity may not positively affect disorders of interest, but whose expression is differentially expressed and contributes to a gene expression fingerprint pattern correlative of, for example, cancer, such pathway genes may additionally be designated as fingerprint genes.

A variety of techniques can be utilized to further characterize the identified genes. First, the nucleotide sequence of the identified genes, which may be obtained by utilizing standard techniques well known to those of skill in the art, may, for example, be used to reveal homologies to one or more known sequence motifs which may yield information regarding the biological function of the identified gene product.

Second, an analysis of the tissue and/or cell type distribution of the mRNA produced by the identified genes may be conducted, utilizing standard techniques well known to those of skill in the art. Such techniques may include, for example, Northern, RNase protection and RT-PCR analyses. Such analyses provide information as to, for example, whether the identified genes are expressed in tissues or cell types expected to contribute to the disorders of interest. Such analyses may also provide quantitative information regarding steady state mRNA regulation, yielding data concerning which of the identified genes exhibits a high level of regulation in, preferably, tissues which may be expected to contribute to the disorders of interest. Additionally, standard in situ hybridization techniques may be utilized to provide information regarding which cells within a given tissue express the identified gene. Such an analysis may provide information regarding the biological function of an identified gene relative to a given disorder in instances wherein only a subset of the cells within the tissue is thought to be relevant to the disorder.

Third, the sequences of the identified genes may be used, utilizing standard techniques, to place the genes onto genetic maps, e.g., mouse (Copeland and Jenkins, Trends in Genetics 7, 113-18, 1991) and human genetic maps (Cohen et al., Nature 366, 698-701, 1993). Such mapping information may yield information regarding the genes' importance to human disease by, for example, identifying genes which map within genetic regions to which known genetic disorders map.

Fourth, the biological function of the identified genes may be more directly assessed by utilizing relevant in vivo and in vitro systems. In vivo systems may include, but are not limited to, animal systems which naturally exhibit symptoms of interest, or ones which have been engineered to exhibit such symptoms. Further, such systems may include systems for the further characterization of a disorder of interest and may include, but are not limited to, naturally occurring and transgenic animal systems. In vitro systems may include, but are not limited to, cell-based systems comprising cell types known or suspected of contributing to the disorder of interest. Such cells may be wild type cells, or may be non-wild type cells containing modifications known to, or suspected of, contributing to the disorder of interest.

In further characterizing the biological function of the identified genes, the expression of these genes may be modulated within the in vitro and/or in vitro systems, i.e., either overexpressed or underexpressed in, for example, transgenic animals and/or cell lines, and its subsequent effect on the system then assayed. Alternatively, the activity of the product of the identified gene may be modulated by either increasing or decreasing the level of activity in the in vivo and/or in vitro system of interest, and its subsequent effect then assayed.

The information obtained through such characterizations may suggest relevant methods for the treatment of disorders involving the gene of interest. Further, relevant methods for the treatment of such disorders involving the gene of interest may be suggested by information obtained from such characterizations. For example, treatment may include a modulation of gene expression and/or gene product activity. Characterization procedures such as those described herein may indicate where such modulation should involve an increase or a decrease in the expression or activity of the gene or gene product of interest.

Screening Methods

The invention provides assays for screening test compounds which bind to or modulate the activity of a MAP kinase phosphatase-like enzyme polypeptide or a MAP kinase phosphatase-like enzyme polynucleotide. A test compound preferably binds to a MAP kinase phosphatase-like enzyme polypeptide or polynucleotide. More preferably, a test compound decreases or increases MAP kinase phosphatase-like enzyme by at least about 10, preferably about 50, more preferably about 75, 90, or 100% relative to the absence of the test compound.

Test Compounds

Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds. See Lam, Anticancer Drug Des. 12, 145, 1997.

Methods for the synthesis of molecular libraries are well known in the art (see, for example, DeWitt et al., Proc. Natl. Acad. Sci. U.S.A. 90, 6909, 1993; Erb et al. Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-421, 1992), or on beads (Lam, Nature 354, 82-84, 1991), chips (Fodor, Nature 364, 555-556, 1993), bacteria or spores (Ladner, U.S. Pat. No. 5,223,409), plasmids (Cull et al., Proc. Natl. Acad. Sci. U.S.A. 89, 1865-1869, 1992), or phage (Scott & Smith, Science 249, 386-390, 1990; Devlin, Science 249, 404-406, 1990); Cwirla et al., Proc. Natl. Acad. Sci. 97, 6378-6382, 1990; Felici, J. Mol. Biol. 222, 301-310, 1991; and Ladner, U.S. Pat. No. 5,223,409).

High Throughput Screening

Test compounds can be screened for the ability to bind to MAP kinase phosphatase-like enzyme polypeptides or polynucleotides or to affect MAP kinase phosphatase-like enzyme activity or MAP kinase phosphatase-like enzyme gene expression using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format.

Alternatively, “free format assays,” or assays that have no physical barrier between samples, can be used. For example, an assay using pigment cells (melanocytes) in a simple homogeneous assay for combinatorial peptide libraries is described by Jayawickreme et al., Proc. Natl. Acad. Sci. U.S.A. 19, 1614-18 (1994). The cells are placed under agarose in petri dishes, then beads that carry combinatorial compounds are placed on the surface of the agarose. The combinatorial compounds are partially released the compounds from the beads. Active compounds can be visualized as dark pigment areas because, as the compounds diffuse locally into the gel matrix, the active compounds cause the cells to change colors.

Another example of a free format assay is described by Chelsky, “Strategies for Screening Combinatorial Libraries: Novel and Traditional Approaches,” reported at the First Annual Conference of The Society for Biomolecular Screening in Philadelphia, Pa. (Nov. 7-10, 1995). Chelsky placed a simple homogenous enzyme assay for carbonic anhydrase inside an agarose gel such that the enzyme in the gel would cause a color change throughout the gel. Thereafter, beads carrying combinatorial compounds via a photolinker were placed inside the gel and the compounds were partially released by UV-light. Compounds that inhibited the enzyme were observed as local zones of inhibition having less color change.

Yet another example is described by Salmon et al., Molecular Diversity 2, 57-63 (1996). In this example, combinatorial libraries were screened for compounds that had cytotoxic effects on cancer cells growing in agar.

Another high throughput screening method is described in Beutel et al., U.S. Pat. No. 5,976,813. In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

Binding Assays

For binding assays, the test compound is preferably a small molecule which binds to and occupies, for example, the ATP/GTP binding site of the enzyme or the active site of the MAP kinase phosphatase-like enzyme polypeptide, such that normal biological activity is prevented. Examples of such small molecules include, but are not limited to, small peptides or peptide-like molecules.

In binding assays, either the test compound or the MAP kinase phosphatase-like enzyme polypeptide can comprise a detectable label, such as a fluorescent, radioisotopic, chemiluminescent, or enzymatic label, such as horseradish peroxidase, alkaline phosphatase, or luciferase. Detection of a test compound which is bound to the MAP kinase phosphatase-like enzyme polypeptide can then be accomplished, for example, by direct counting of radioemmission, by scintillation counting, or by determining conversion of an appropriate substrate to a detectable product.

Alternatively, binding of a test compound to a MAP kinase phosphatase-like enzyme polypeptide can be determined without labeling either of the interactants. For example, a microphysiometer can be used to detect binding of a test compound with a MAP kinase phosphatase-like enzyme polypeptide. A microphysiometer (e.g., Cytosensor™) is an analytical instrument that measures the rate at which a cell acidifies its environment using a light-addressable potentiometric sensor (LAPS). Changes in this acidification rate can be used as an indicator of the interaction between a test compound and a MAP kinase phosphatase-like enzyme polypeptide (McConnell et al., Science 257, 1906-1912, 1992).

Determining the ability of a test compound to bind to a MAP kinase phosphatase-like enzyme polypeptide also can be accomplished using a technology such as real-time Bimolecular Interaction Analysis (BIA) (Sjolander & Urbaniczky, Anal. Chem. 63, 2338-2345, 1991, and Szabo et al., Curr. Opin. Struct. Biol. 5, 699-705, 1995). BIA is a technology for studying biospecific interactions in real time, without labeling any of the interactants (e.g., BIAcore™). Changes in the optical phenomenon surface plasmon resonance (SPR) can be used as an indication of real-time reactions between biological molecules.

In yet another aspect of the invention, a MAP kinase phosphatase-like enzyme polypeptide can be used as a “bait protein” in a two-hybrid assay or three-hybrid assay (see, e.g., U.S. Pat. No. 5,283,317; Zervos et al., Cell 72, 223-232, 1993; Madura et al., J. Biol. Chem. 268, 12046-12054, 1993; Bartel et al., BioTechniques 14, 920-924, 1993; Iwabuchi et al., Oncogene 8, 1693-1696, 1993; and Brent W094/10300), to identify other proteins which bind to or interact with the MAP kinase phosphatase-like enzyme polypeptide and modulate its activity.

The two-hybrid system is based on the modular nature of most transcription factors, which consist of separable DNA-binding and activation domains. Briefly, the assay utilizes two different DNA constructs. For example, in one construct, polynucleotide encoding a MAP kinase phosphatase-like enzyme polypeptide can be fused to a polynucleotide encoding the DNA binding domain of a known transcription factor (e.g., GAL-4). In the other construct a DNA sequence that encodes an unidentified protein (“prey” or “sample”) can be fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein which interacts with the MAP kinase phosphatase-like enzyme polypeptide.

It may be desirable to immobilize either the MAP kinase phosphatase-like enzyme polypeptide (or polynucleotide) or the test compound to facilitate separation of bound from unbound forms of one or both of the interactants, as well as to accommodate automation of the assay. Thus, either the MAP kinase phosphatase-like enzyme polypeptide (or polynucleotide) or the test compound can be bound to a solid support. Suitable solid supports include, but are not limited to, glass or plastic slides, tissue culture plates, microtiter wells, tubes, silicon chips, or particles such as beads (including, but not limited to, latex, polystyrene, or glass beads). Any method known in the art can be used to attach the enzyme polypeptide (or polynucleotide) or test compound to a solid support, including use of covalent and non-covalent linkages, passive absorption, or pairs of binding moieties attached respectively to the polypeptide (or polynucleotide) or test compound and the solid support. Test compounds are preferably bound to the solid support in an array, so that the location of individual test compounds can be tracked. Binding of a test compound to a v enzyme polypeptide (or polynucleotide) can be accomplished in any vessel suitable for containing the reactants. Examples of such vessels include microtiter plates, test tubes, and microcentrifuge tubes.

In one embodiment, the MAP kinase phosphatase-like enzyme polypeptide is a fusion protein comprising a domain that allows the MAP kinase phosphatase-like enzyme polypeptide to be bound to a solid support. For example, glutathione-S-transferase fusion proteins can be adsorbed onto glutathione sepharose beads (Sigma Chemical, St. Louis, Mo.) or glutathione derivatized microtiter plates, which are then combined with the test compound or the test compound and the non-adsorbed MAP kinase phosphatase-like enzyme polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either a MAP kinase phosphatase-like enzyme polypeptide (or polynucleotide) or a test compound can be immobilized utilizing conjugation of biotin and streptavidin. Biotinylated MAP kinase phosphatase-like enzyme polypeptides (or polynucleotides) or test compounds can be prepared from biotin-NHS(N-hydroxysuccinimide) using techniques well known in the art (e.g., biotinylation kit, Pierce Chemicals, Rockford, Ill.) and immobilized in the wells of streptavidin-coated 96 well plates Pierce Chemical). Alternatively, antibodies which specifically bind to a MAP kinase phosphatase-like enzyme polypeptide, polynucleotide, or a test compound, but which do not interfere with a desired binding site, such as the active site of the enzyme, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to the MAP kinase phosphatase-like enzyme polypeptide or test compound, enzyme-linked assays which rely on detecting an activity of the MAP kinase phosphatase-like enzyme polypeptide, and SDS gel electrophoresis under non-reducing conditions.

Screening for test compounds which bind to a MAP kinase phosphatase-like enzyme polypeptide or polynucleotide also can be carried out in an intact cell. Any cell which comprises a MAP kinase phosphatase-like enzyme polypeptide or polynucleotide can be used in a cell-based assay system. A MAP kinase phosphatase-like enzyme polynucleotide can be naturally occurring in the cell or can be introduced using techniques such as those described above. Binding of the test compound to a MAP kinase phosphatase-like enzyme polypeptide or polynucleotide is determined as described above.

Enzyme Assays

Test compounds can be tested for the ability to increase or decrease the sphingosine kinase activity of a human MAP kinase phosphatase-like enzyme polypeptide. Enzyme activity can be measured, for example, as described in the specific examples, below.

Enzyme assays can be carried out after contacting either a purified MAP kinase phosphatase-like enzyme polypeptide, a cell membrane preparation, or an intact cell with a test compound. A test compound which decreases activity of a MAP kinase phosphatase-like enzyme polypeptide by at least about 10, preferably about 50, more preferably about 75, 90, or 100% is identified as a potential therapeutic agent for decreasing MAP kinase phosphatase-like enzyme activity. A test compound which increases activity of a human MAP kinase phosphatase-like enzyme polypeptide by at least about 10, preferably about 50, more preferably about 75, 90, or 100% is identified as a potential therapeutic agent for increasing human MAP kinase phosphatase-like enzyme activity.

Gene Expression

In another embodiment, test compounds which increase or decrease MAP kinase phosphatase-like enzyme gene expression are identified. A MAP kinase phosphatase-like enzyme polynucleotide is contacted with a test compound, and the expression of an RNA or polypeptide product of the v enzyme polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of v enzyme mRNA or polypeptide expression in the cells can be determined by methods well known in the art for detecting mRNA or polypeptide. Either qualitative or quantitative methods can be used. The presence of polypeptide products of a MAP kinase phosphatase-like enzyme polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into a MAP kinase phosphatase-like enzyme polypeptide.

Such screening can be carried out either in a cell-free assay system or in an intact cell. Any cell which expresses a MAP kinase phosphatase-like enzyme polynucleotide can be used in a cell-based assay system. The MAP kinase phosphatase-like enzyme polynucleotide can be naturally occurring in the cell or can be introduced using techniques such as those described above. Either a primary culture or an established cell line, such as CHO or human embryonic kidney 293 cells, can be used.

Pharmaceutical Compositions

The invention also provides pharmaceutical compositions which can be administered to a patient to achieve a therapeutic effect. Pharmaceutical compositions of the invention can comprise, for example, a MAP kinase phosphatase-like enzyme polypeptide, MAP kinase phosphatase-like enzyme polynucleotide, ribozymes or antisense oligonucleotides, antibodies which specifically bind to a MAP kinase phosphatase-like enzyme polypeptide, or mimetics, agonists, antagonists, or inhibitors of a MAP kinase phosphatase-like enzyme polypeptide activity. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones.

In addition to the active ingredients, these pharmaceutical compositions can contain suitable pharmaceutically-acceptable carriers comprising excipients and auxiliaries which facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also can contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifing, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation can be a lyophilized powder which can contain any or all of the following: 1-50 mM histidine, 0.1%-2% sucrose, and 2-7% mannitol, at a pH range of 4.5 to 5.5, that is combined with buffer prior to use.

Further details on techniques for formulation and administration can be found in the latest edition of REMINGTON'S PHARMACEUTICAL SCIENCES (Maack Publishing Co., Easton, Pa.). After pharmaceutical compositions have been prepared, they can be placed in an appropriate container and labeled for treatment of an indicated condition. Such labeling would include amount, frequency, and method of administration.

Therapeutic Indications and Methods

Allergies and Asthma

Human MAP kinase phosphatase-like enzyme can be regulated to treat allergies including asthma. Allergy is a complex process in which environmental antigens induce clinically adverse reactions. The inducing antigens, called allergens, typically elicit a specific IgE response and, although in most cases the allergens themselves have little or no intrinsic toxicity, they induce pathology when the IgE response in turn elicits an IgE-dependent or T cell-dependent hypersensitivity reaction. Hypersensitivity reactions can be local or systemic and typically occur within minutes of allergen exposure in individuals who have previously been sensitized to an allergen. The hypersensitivity reaction of allergy develops when the allergen is recognized by IgE antibodies bound to specific receptors on the surface of effector cells, such as mast cells, basophils, or eosinophils, which causes the activation of the effector cells and the release of mediators that produce the acute signs and symptoms of the reactions. Allergic diseases include asthma, allergic rhinitis (hay fever), atopic dermatitis, and anaphylaxis.

Asthma is though to arise as a result of interactions between multiple genetic and environmental factors and is characterized by three major features: 1) intermittent and reversible airway obstruction caused by bronchoconstriction, increased mucus production, and thickening of the walls of the airways that leads to a narrowing of the airways, 2) airway hyperresponsiveness caused by a decreased control of airway caliber, and 3) airway inflammation. Certain cells are critical to the inflammatory reaction of asthma and they include T cells and antigen presenting cells, B cells that produce IgE, and mast cells, basophils, eosinophils, and other cells that bind IgE. These effector cells accumulate at the site of allergic reaction in the airways and release toxic products that contribute to the acute pathology and eventually to the tissue destruction related to the disorder. Other resident cells, such as smooth muscle cells, lung epithelial cells, mucus-producing cells, and nerve cells may also be abnormal in individuals with asthma and may contribute to the pathology. While the airway obstruction of asthma, presenting clinically as an intermittent wheeze and shortness of breath, is generally the most pressing symptom of the disease requiring immediate treatment, the inflammation and tissue destruction associated with the disease can lead to irreversible changes that eventually make asthma a chronic disabling disorder requiring long-term management.

Despite recent important advances in our understanding of the pathophysiology of asthma, the disease appears to be increasing in prevalence and severity (Gergen and Weiss, Am Rev Respir Dis 146:823-824, 1992). It is estimated that 30-40% of the population suffer with atopic allergy, and 15% of children and 5% of adults in the population suffer from asthma (Gergen and Weiss, Am Rev Respir Dis 146:823-824, 1992). Thus, an enormous burden is placed on our health care resources. However, both diagnosis and treatment of asthma are difficult. The severity of lung tissue inflammation is not easy to measure and the symptoms of the disease are often indistinguishable from those of respiratory infections, chronic respiratory inflammatory disorders, allergic rhinitis, or other respiratory disorders. Often, the inciting allergen cannot be determined, making removal of the causative environmental agent difficult. Current pharmacological treatments suffer their own set of disadvantages. Commonly used therapeutic agents, such as beta agonists, can act as symptom relievers to transiently improve pulmonary function, but do not affect the underlying inflammation. Agents that can reduce the underlying inflammation, such as anti-inflammatory steroids, can have major drawbacks that range from immunosuppression to bone loss (Goodman and Gilman's The Pharmacologic Basis of Therapeutics, Seventh Edition, MacMillan Publishing Company, NY, USA, 1985). In addition, many of the present therapies, such as inhaled corticosteroids, are short-lasting, inconvenient to use, and must be used often on a regular basis, in some cases for life, making failure of patients to comply with the treatment a major problem and thereby reducing their effectiveness as a treatment.

Because of the problems associated with conventional therapies, alternative treatment strategies have been evaluated. Glycophorin A (Chu and Sharom, Cell Immunol 145:223-239, 1992), cyclosporin (Alexander et al., Lancet 1992, 339:324-328) and a nonapeptide fragment of IL-2 (Zav'yalov et al. Immunol Lett 1992, 31:285-288) all inhibit interleukin-2 dependent T lymphocyte proliferation; however, they are known to have many other effects. For example, cyclosporin is used as a immuno-suppressant after organ transplantation. While these agents may represent alternatives to steroids in the treatment of asthmatics, they inhibit interleukin-2 dependent T lymphocyte proliferation and potentially critical immune functions associated with homeostasis. Other treatments that block the release or activity of mediators of bronchochonstriction, such as cromones or anti-leukotrienes, have recently been introduced for the treatment of mild asthma, but they are expensive and not effective in all patients and it is unclear whether they have any effect on the chronic changes associated with asthmatic inflammation. What is needed in the art is the identification of a treatment that can act in pathways critical to the development of asthma that both blocks the episodic attacks of the disorder and preferentially dampens the hyperactive allergic immune response without immunocompromising the patient.

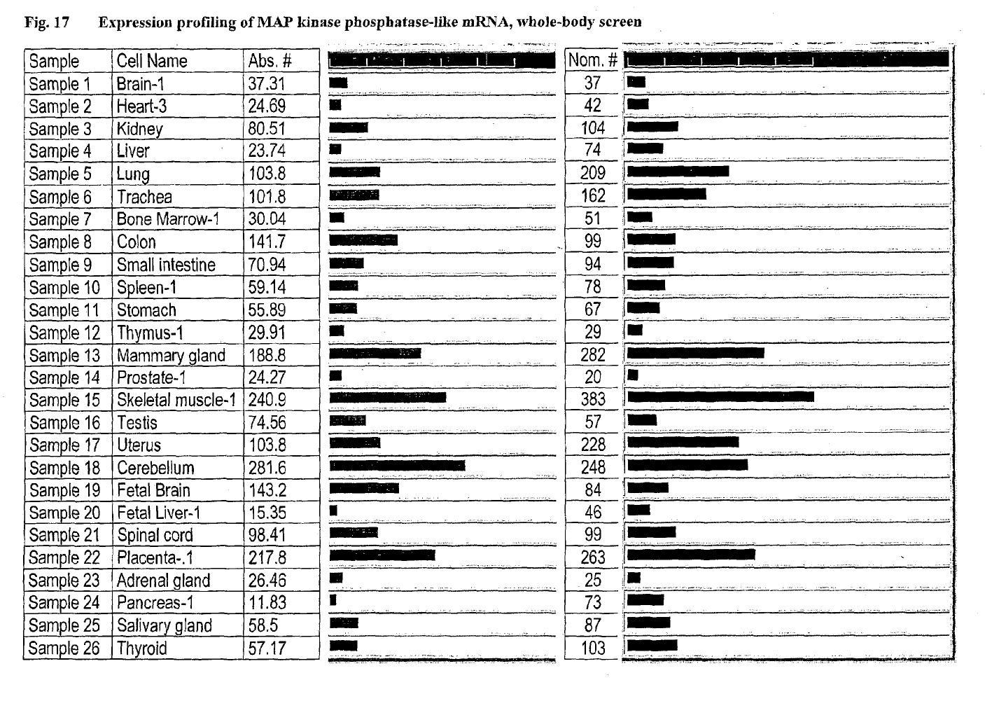

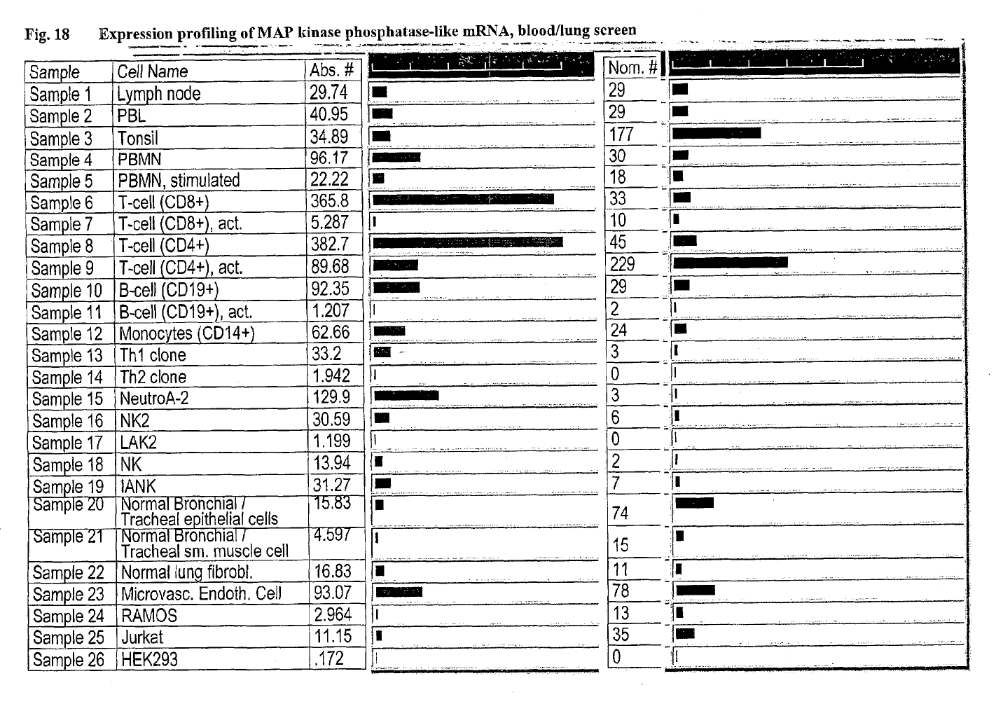

MAP kinase phosphatase-like enzyme shows high expression in skeletal muscle, mammary gland, placenta, cerebellum, uterus, lung, tonsil, and trachea, and moderate expression in most other organs (FIG. 17). Among cell types tested, MAP kinase phosphatase-like enzyme shows high expression only in activated CD4⁺ T cells (FIG. 18). Different members of the family of dual specificity phosphatases, to which MAP kinase phosphatase-like enzyme is predicted to belong, show distinct substrate specificities for various MAP kinases, different tissue distribution, and different modes of inducibility of their expression by extracellular stimuli. The pattern of tissue expression seen for MAP kinase phosphatase-like enzyme is therefore expected to resemble that of its specific MAP kinase substrate, which is as yet unknown. Its high expression in mammary gland, placenta, and uterus indicates that either its expression or the expression of its subtrate may be induced by sex hormones. Its high expression in respiratory tract tissues, tonsils, and activated CD4⁺ T cells, on the other hand, suggests that it plays an important role in the defense against airborne pathogens and toxins and in the response against airborne allergens.

CNS Disorders

Human MAP kinase phosphatase-like enzyme can be regulated to treat CNS disorders. Because apoptosis in neuronal cells contributes to the morbidity associated with neurodegenerative diseases, stroke and Alzheimer's dementia (84), the enzyme of the present invention may be used in the development of novel therapeutic strategies for neurodegenerative diseases. CNS disorders which can be treated include brain injuries, cerebrovascular diseases and their consequences, Parkinson's disease, corticobasal degeneration, motor neuron disease, dementia, including ALS, multiple sclerosis, traumatic brain injury, stroke, post-stroke, post-traumatic brain injury, and small-vessel cerebrovascular disease. Dementias, such as Alzheimer's disease, vascular dementia, dementia with Lewy bodies, frontotemporal dementia and Parkinsonism linked to chromosome 17, frontotemporal dementias, including Pick's disease, progressive nuclear palsy, corticobasal degeneration, Huntington's disease, thalamic degeneration, Creutzfeld-Jakob dementia, HIV dementia, schizophrenia with dementia, and Korsalroff's psychosis also can be treated. Similarly, it is possible to treat cognitive-related disorders, such as mild cognitive impairment, age-associated memory impairment, age-related cognitive decline, vascular cognitive impairment, attention deficit disorders, attention deficit hyperactivity disorders, and memory disturbances in children with learning disabilities, by regulating the activity of human MAP kinase phosphatase-like enzyme.

Diabetes

In rats, protein expression of MAP kinase phosphatase-1 is decreased in diabetes mellitus relative to control levels, which may have implications for the pathogenesis of diabetic nephropathy. Awazu et al., J. Am1994. Soc. Nephrol. 10, 738-45, 1999. Human MAP kinase phosphatase-like enzyme, therefore, is likely to be useful for treatment of diabetes. Diabetes mellitus is a common metabolic disorder characterized by an abnormal elevation in blood glucose, alterations in lipids and abnormalities (complications) in the cardiovascular system, eye, kidney and nervous system. Diabetes is divided into two separate diseases: type 1 diabetes juvenile onset), which results from a loss of cells which make and secrete insulin, and type 2 diabetes (adult onset), which is caused by a defect in insulin secretion and a defect in insulin action.

Type 1 diabetes is initiated by an autoimuune reaction that attacks the insulin secreting cells (beta cells) in the pancreatic islets. Agents that prevent this reaction from occurring or that stop the reaction before destruction of the beta cells has been accomplished are potential therapies for this disease. Other agents that induce beta cell proliferation and regeneration also are potential therapies.

Type II diabetes is the most common of the two diabetic conditions (6% of the population). The defect in insulin secretion is an important cause of the diabetic condition and results from an inability of the beta cell to properly detect and respond to rises in blood glucose levels with insulin release. Therapies that increase the response by the beta cell to glucose would offer an important new treatment for this disease.

The defect in insulin action in Type II diabetic subjects is another target for therapeutic intervention. Agents that increase the activity of the insulin receptor in muscle, liver, and fat will cause a decrease in blood glucose and a normalization of plasma lipids. The receptor activity can be increased by agents that directly stimulate the receptor or that increase the intracellular signals from the receptor. Other therapies can directly activate the cellular end process, i.e. glucose transport or various enzyme systems, to generate an insulin-like effect and therefore a produce beneficial outcome. Because overweight subjects have a greater susceptibility to Type II diabetes, any agent that reduces body weight is a possible therapy.

Both Type I and Type diabetes can be treated with agents that mimic insulin action or that treat diabetic complications by reducing blood glucose levels. Likewise, agents that reduces new blood vessel growth can be used to treat the eye complications that develop in both diseases.

Obesity

Human MAP kinase phosphatase-like enzyme can be regulated to treat obesity. Obesity and overweight are defined as an excess of body fat relative to lean body mass. An increase in caloric intake or a decrease in energy expenditure or both can bring about this imbalance leading to surplus energy being stored as fat. Obesity is associated with important medical morbidities and an increase in mortality. The causes of obesity are poorly understood and may be due to genetic factors, environmental factors or a combination of the two to cause a positive energy balance. In contrast, anorexia and cachexia are characterized by an imbalance in energy intake versus energy expenditure leading to a negative energy balance and weight loss. Agents that either increase energy expenditure and/or decrease energy intake, absorption or storage would be useful for treating obesity, overweight, and associated comorbidities. Agents that either increase energy intake and/or decrease energy expenditure or increase the amount of lean tissue would be useful for treating cachexia, anorexia and wasting disorders.

This gene, translated proteins and agents which modulate this gene or portions of the gene or its products are useful for treating obesity, overweight, anorexia, cachexia, wasting disorders, appetite suppression, appetite enhancement, increases or decreases in satiety, modulation of body weight, and/or other eating disorders such as bulimia. Also this gene, translated proteins and agents which modulate this gene or portions of the gene or its products are useful for treating obesity/overweight-associated comorbidities including hypertension, type 2 diabetes, coronary artery disease, hyperlipidemia, stroke, gallbladder disease, gout, osteoarthritis, sleep apnea and respiratory problems, some types of cancer including endometrial, breast, prostate, and colon cancer, thrombolic disease, polycystic ovarian syndrome, reduced fertility, complications of pregnancy, menstrual irregularities, hirsutism, stress incontinence, and depression.

Chronic Obstructive Pulmonary Disease

Human MAP kinase phosphatase-like enzyme can be regulated to treat chronic obstructive pulmonary disease. Chronic obstructive pulmonary (or airways) disease (COPD) is a condition defined physiologically as airflow obstruction that generally results from a mixture of emphysema and peripheral airway obstruction due to chronic bronchitis (Senior & Shapiro, Pulmonary Diseases and Disorders, 3d ed., New York, McGraw-Hill, 1998, pp. 659-681, 1998; Barnes, Chest 117, 10S-14S, 2000). Emphysema is characterized by destruction of alveolar walls leading to abnormal enlargement of the air spaces of the, lung. Chronic bronchitis is defined clinically as the presence of chronic productive cough for three months in each of two successive years. In COPD, airflow obstruction is usually progressive and is only partially reversible. By far the most important risk factor for development of COPD is cigarette smoking, although the disease does occur in non-smokers.

Chronic inflammation of the airways is a key pathological feature of COPD (Senior & Shapiro, 1998). The inflammatory cell population comprises increased numbers of macrophages, neutrophils, and CD8⁺ lymphocytes. Inhaled irritants, such as cigarette smoke, activate macrophages which are resident in the respiratory tract, as well as epithelial cells leading to release of chemokines (e.g., interleukin-8) and other chemotactic factors. These chemotactic factors act to increase the neutrophil/-monocyte trafficking from the blood into the lung tissue and airways. Neutrophils and monocytes recruited into the airways can release a variety of potentially damaging mediators such as proteolytic enzymes and reactive oxygen species.

Matrix degradation and emphysema, along with airway wall thickening, surfactant dysfunction, and mucus hypersecretion, all are potential sequelae of this inflammatory response that lead to impaired airflow and gas exchange.

Cardiovascular Diseases

MAP kinase phosphatase-1 is decreased after injury to the rat carotid artery and may be partially responsible for proliferation of smooth muscle cells after vascular injury. Lai et al., J. Clin. Invest. 98, 1560-67, 1996. Human MAP kinase phosphatase-like enzyme, therefore, is likely to be useful for treatment of cardiovascular diseases.

Cardiovascular diseases include the following disorders of the heart and the vascular system: congestive heart failure, myocardial infarction, ischemic diseases of the heart, all kinds of atrial and ventricular arrhythmias, hypertensive vascular diseases, and peripheral vascular diseases.

Heart failure is defined as a pathophysiologic state in which an abnormality of cardiac function is responsible for the failure of the heart to pump blood at a rate commensurate with the requirement of the metabolizing tissue. It includes all forms of pumping failure, such as high-output and low-output, acute and chronic, right-sided or left-sided, systolic or diastolic, independent of the underlying cause.

Myocardial infarction (MI) is generally caused by an abrupt decrease in coronary blood flow that follows a thrombotic occlusion of a coronary artery previously narrowed by arteriosclerosis. MI prophylaxis (primary and secondary prevention) is included, as well as the acute treatment of MI and the prevention of complications.

Ischemic diseases are conditions in which the coronary flow is restricted resulting in a perfusion which is inadequate to meet the myocardial requirement for oxygen. This group of diseases includes stable angina, unstable angina, and asymptomatic ischemia.

Arrhythmias include all forms of atrial and ventricular tachyarrhythmias (atrial tachycardia, atrial flutter, atrial fibrillation, atrio-ventricular reentrant tachycardia, preexcitation syndrome, ventricular tachycardia, ventricular flutter, and ventricular fibrillation), as well as bradycardic forms of arrhythmias.

Hypertensive vascular diseases include primary as well as all kinds of secondary arterial hypertension (renal, endocrine, neurogenic, others). The disclosed gene and its product may be used as drug targets for the treatment of hypertension as well as for the prevention of all complications.

Peripheral vascular diseases are defined as vascular diseases in which arterial and/or venous flow is reduced resulting in an imbalance between blood supply and tissue oxygen demand. It includes chronic peripheral arterial occlusive disease (PAOD), acute arterial thrombosis and embolism, inflammatory vascular disorders, Raynaud's phenomenon, and venous disorders.

Cancer

Human MAP kinase phosphatase-like enzyme and inhibitors thereof can be used in therapeutic applications such as cancer therapy. Cancer is a disease fundamentally caused by oncogenic cellular transformation. There are several hallmarks of transformed cells that distinguish them from their normal counterparts and underlie the pathophysiology of cancer. These include uncontrolled cellular proliferation, unresponsiveness to normal death-inducing signals (imnmortalization), increased cellular motility and invasiveness, increased ability to recruit blood supply through induction of new blood vessel formation (angiogenesis), genetic instability, and dysregulated gene expression. Various combinations of these aberrant physiologies, along with the acquisition of drug-resistance frequently lead to an intractable disease state in which organ failure and patient death ultimately ensue.

Most standard cancer therapies target cellular proliferation and rely on the differential proliferative capacities between transformed and normal cells for their efficacy. This approach is hindered by the facts that several important normal cell types are also highly proliferative and that cancer cells frequently become resistant to these agents. Thus, the therapeutic indices for traditional anti-cancer therapies rarely exceed 2.0.

The advent of genomics-driven molecular target identification has opened up the possibility of identifying new cancer-specific targets for therapeutic intervention that will provide safer, more effective treatments for cancer patients. Thus, newly discovered tumor-associated genes and their products can be tested for their role(s) in disease and used as tools to discover and develop innovative therapies. Genes playing important roles in any of the physiological processes outlined above can be characterized as cancer targets.

Genes or gene fragments identified through genomics can readily be expressed in one or more heterologous expression systems to produce functional recombinant proteins. These proteins are characterized in vitro for their biochemical properties and then used as tools in high-throughput molecular screening programs to identify chemical modulators of their biochemical activities. Agonists and/or antagonists of target protein activity can be identified in this manner and subsequently tested in cellular and in vivo disease models for anti-cancer activity. Optimization of lead compounds with iterative testing in biological models and detailed pharmacokinetic and toxicological analyses form the basis for drug development and subsequent testing in humans.

Expression of MKPs in human tumors including prostate, breast, colon, lung and bladder, function to direct oncogenic signals into a proliferative pathway away from apoptosis. For example, MAP protein kinase phosphatase 1 is overexpressed in prostate cancers and is inversely related to apoptosis. Magi-Galluzzi et al., Laboratory Invest. 76, 37-51, 1997. The use of inhibitors of the MAP kinase phosphatase-like enzyme of the present invention, will selectively act on tumor cells to redirect the oncogenic signal into apoptopic pathways. It will be appreciated that such novel chemotherapeutics may be combined with current non-surgical treatment for human cancers including radiation and chemotherapy, both of which are stimulators of the stress-activated protein kinase cascade (79, 85) that kill tumor cells by triggering apoptosis (86).

This invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense nucleic acid molecule, a specific antibody, ribozyme, or a MAP kinase phosphatase-like enzyme polypeptide binding molecule) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

A reagent which affects MAP kinase phosphatase-like enzyme activity can be administered to a human cell, either in vitro or in vivo, to reduce MAP kinase phosphatase-like enzyme activity. The reagent preferably binds to an expression product of a human MAP kinase phosphatase-like enzyme gene. If the expression product is a protein, the reagent is preferably an antibody. For treatment of human cells ex vivo, an antibody can be added to a preparation of stem cells which have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung, liver, spleen, heart brain, lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 10⁶ cells, more preferably about 1.0 μg of DNA per 16 nmole of liposome delivered to about 10⁶ cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

Complexing a liposome with a reagent such as an antisense oligonucleotide or ribozyme can be achieved using methods which are standard in the art (see, for example, U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 nmol liposomes, and even more preferably about 1.0 μg of polynucleotides is combined with about 8 nmol liposomes.

In another embodiment, antibodies can be delivered to specific tissues in vivo using receptor-mediated targeted delivery. Receptor-mediated DNA delivery techniques are taught in, for example, Findeis et al. Trends in Biotechnol. 11, 202-05 (1993); Chiou et al., GENE THERAPEUTICS: METHODS AND APPLICATIONS OF DIRECT GENE TRANSFER (J. A. Wolff, ed.) (1994); Wu & Wu, J. Biol. Chem. 263, 621-24 (1988); Wu et al., J. Biol. Chem. 269, 542-46 (1994); Zenke et al., Proc. Natl. Acad. Sci. U.S.A. 87, 3655-59 (1990); Wu et al., J. Biol. Chem. 266, 338-42 (1991).

Determination of a Therapeutically Effective Dose

The determination of a therapeutically effective dose is well within the capability of those skilled in the art. A therapeutically effective dose refers to that amount of active ingredient which increases or decreases kinase-like enzyme activity relative to the MAP kinase phosphatase-like enzyme activity which occurs in the absence of the therapeutically effective dose.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Normal dosage amounts can vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

If the reagent is a single-chain antibody, polynucleotides encoding the antibody can be constructed and introduced into a cell either ex vivo or in vivo using well-established techniques including, but not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and DEAE- or calcium phosphate-mediated transfection.

Effective in vivo dosages of an antibody are in the range of about 5 μg to about 50 μg/kg, about 50 μg to about 5 mg/kg, about 100 μg to about 500 μg/kg of patient body weight, and about 200 to about 250 μg/kg of patient body weight. For administration of polynucleotides encoding single-chain antibodies, effective in vivo dosages are in the range of about 100 ng to about 200 ng, 500 ng to about 50 mg, about 1 μg to about 2 mg, about 5 μg to about 500 μg, and about 20 μg to about 100 μg of DNA.

If the expression product is mRNA, the reagent is preferably an antisense oligonucleotide or a ribozyme. Polynucleotides which express antisense oligonucleotides or ribozymes can be introduced into cells by a variety of methods, as described above.

Preferably, a reagent reduces expression of a MAP kinase phosphatase-like enzyme gene or the activity of a MAP kinase phosphatase-like enzyme polypeptide by at least about 10, preferably about 50, more preferably about 75, 90, or 100% relative to the absence of the reagent. The effectiveness of the mechanism chosen to decrease the level of expression of a MAP kinase phosphatase-like enzyme gene or the activity of a MAP kinase phosphatase-like enzyme polypeptide can be assessed using methods well known in the art, such as hybridization of nucleotide probes to MAP kinase phosphatase-like enzyme-specific mRNA, quantitative RT-PCR, immunologic detection of a MAP kinase phosphatase-like enzyme polypeptide, or measurement of MAP kinase phosphatase-like enzyme activity.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects.

Any of the therapeutic methods described above can be applied to any subject in need of such therapy, including, for example, mammals such as dogs, cats, cows, horses, rabbits, monkeys, and most preferably, humans.

Diagnostic Methods

Human MAP kinase phosphatase-like enzyme also can be used in diagnostic assays for detecting diseases and abnormalities or susceptibility to diseases and abnormalities related to the presence of mutations in the nucleic acid sequences which encode the enzyme. For example, differences can be determined between the cDNA or genomic sequence encoding MAP kinase phosphatase-like enzyme in individuals afflicted with a disease and in normal individuals. If a mutation is observed in some or all of the afflicted individuals but not in normal individuals, then the mutation is likely to be the causative agent of the disease.

Sequence differences between a reference gene and a gene having mutations can be revealed by the direct DNA sequencing method. In addition, cloned DNA segments can be employed as probes to detect specific DNA segments. The sensitivity of this method is greatly enhanced when combined with PCR. For example, a sequencing primer can be used with a double-stranded PCR product or a single-stranded template molecule generated by a modified PCR. The sequence determination is performed by conventional procedures using radiolabeled nucleotides or by automatic sequencing procedures using fluorescent tags.

Genetic testing based on DNA sequence differences can be carried out by detection of alteration in electrophoretic mobility of DNA fragments in gels with or without denaturing agents. Small sequence deletions and insertions can be visualized, for example, by high resolution gel electrophoresis. DNA fragments of different sequences can be distinguished on denaturing formamide gradient gels in which the mobilities of different DNA fragments are retarded in the gel at different positions according to their specific melting or partial melting temperatures (see, e.g., Myers et al., Science 230, 1242, 1985). Sequence changes at specific locations can also be revealed by nuclease protection assays, such as RNase and S 1 protection or the chemical cleavage method (e.g., Cotton et al., Proc. Natl. Acad. Sci. USA 85, 4397-4401, 1985). Thus, the detection of a specific DNA sequence can be performed by methods such as hybridization, RNase protection, chemical cleavage, direct DNA sequencing or the use of restriction enzymes and Southern blotting of genomic DNA. In addition to direct methods such as gel-electrophoresis and DNA sequencing, mutations can also be detected by in situ analysis.

Altered levels of a MAP kinase phosphatase-like enzyme also can be detected in various tissues. Assays used to detect levels of the receptor polypeptides in a body sample, such as blood or a tissue biopsy, derived from a host are well known to those of skill in the art and include radioimmunoassays, competitive binding assays, Western blot analysis, and ELISA assays.

All patents and patent applications cited in this disclosure are expressly incorporated herein by reference. The above disclosure generally describes the present invention. A more complete understanding can be obtained by reference to the following specific examples which are provided for purposes of illustration only and are not intended to limit the scope of the invention.

### EXAMPLE 1

Detection of MAP Kinase Phosphatase-Like Enzyme Activity

The polynucleotide of SEQ ID NO: 1 or 10 is inserted into the expression vector pCEV4 and the expression vector pCEV4-MAP kinase phosphatase-like enzyme polypeptide obtained is transfected into human embryonic kidney 293 cells. From these cells extracts are obtained and the MAP kinase phosphatase-like enzyme activity is measured at 37° C. using p-nitrophenyl phosphate (pNPP) (Sigma) as a substrate. Reactions are performed for 15 min in 200 μl of 50 mM imidazole (pH 7,5) containing 10 mM dithiothreitol, 20 mM pNPP, and the indicated amounts of the cell extract. The reaction is stopped by the addition of 0,1 N NaOH, and the pNPP hydrolyzed is measured by absorbance at 405 nm with a microplate reader (Life Technologies, Inc.). It is shown that the polypeptide of SEQ ID NO: 2 and 11 has a MAP kinase phosphatase-like enzyme activity.

### EXAMPLE 2

Expression of Recombinant Human MAP Kinase Phosphatase-Like Enzyme

The Pichia pastoris expression vector pPICZB (Invitrogen, San Diego, calif.) is used to produce large quantities of recombinant human MAP kinase phosphatase-like enzyme polypeptides in yeast. The MAP kinase phosphatase-like enzyme-encoding DNA sequence is derived from SEQ ID NO:1 or 10. Before insertion into vector pPICZB, the DNA sequence is modified by well known methods in such a way that it contains at its 5′-end an initiation codon and at its 3′-end an enterokinase cleavage site, a His6 reporter tag and a termination codon. Moreover, at both termini recognition sequences for restriction endonucleases are added and after digestion of the multiple cloning site of pPICZ B with the corresponding restriction enzymes the modified DNA sequence is ligated into pPICZB. This expression vector is designed for inducible expression in Pichia pastoris, driven by a yeast promoter. The resulting pPICZ/md-His6 vector is used to transform the yeast.

The yeast is cultivated under usual conditions in 5 liter shake flasks and the recombinantly produced protein isolated from the culture by affinity chromatography (Ni-NTA-Resin) in the presence of 8 M urea. The bound polypeptide is eluted with buffer, pH 3.5, and neutralized. Separation of the polypeptide from the His6 reporter tag is accomplished by site-specific proteolysis using enterokinase (Invitrogen, San Diego, calif.) according to manufacturer's instructions. Purified human MAP kinase phosphatase-like enzyme polypeptide is obtained.

### EXAMPLE 3

Identification of Test Compounds that Bind to MAP Kinase Phosphatase-Like Enzyme Polypeptides

Purified MAP kinase phosphatase-like enzyme polypeptides comprising a glutathione-S-transferase protein and absorbed onto glutathione-derivatized wells of 96-well microtiter plates are contacted with test compounds from a small molecule library at pH 7.0 in a physiological buffer solution. Human MAP kinase phosphatase-like enzyme polypeptides comprise the amino acid sequence shown in SEQ ID NO:2 or 11. The test compounds comprise a fluorescent tag. The samples are incubated for 5 minutes to one hour. Control samples are incubated in the absence of a test compound.

The buffer solution containing the test compounds is washed from the wells. Binding of a test compound to a MAP kinase phosphatase-like enzyme polypeptide is detected by fluorescence measurements of the contents of the wells. A test compound which increases the fluorescence in a well by at least 15% relative to fluorescence of a well in which a test compound is not incubated is identified as a compound which binds to a MAP kinase phosphatase-like enzyme polypeptide.

### EXAMPLE 4

Identification of a Test Compound which Decreases MAP Kinase Phosphatase-Like Enzyme Gene Expression

A test compound is administered to a culture of human cells transfected with a MAP kinase phosphatase-like enzyme expression construct and incubated at 37° C. for 10 to 45 minutes. A culture of the same type of cells which have not been transfected is incubated for the same time without the test compound to provide a negative control.

RNA is isolated from the two cultures as described in Chirgwin et al., Biochem. 18, 5294-99, 1979). Northern blots are prepared using 20 to 30 μg total RNA and hybridized with a ³²P-labeled MAP kinase phosphatase-like enzyme-specific probe at 65° C. in Express-hyb (CLONTECH). The probe comprises at least 11 contiguous nucleotides selected from the complement of SEQ ID NO:1 or 10. A test compound which decreases the MAP kinase phosphatase-like enzyme-specific signal relative to the signal obtained in the absence of the test compound is identified as an inhibitor of MAP kinase phosphatase-like enzyme gene expression.

### EXAMPLE 5

Identification of a Test Compound which Decreases MAP Kinase Phosphatase-Like Enzyme Activity

A test compound is administered to a culture of human cells transfected with a MAP kinase phosphatase-like enzyme expression construct and incubated at 37° C. for 10 to 45 minutes. A culture of the same type of cells which have not been transfected is incubated for the same time without the test compound to provide a negative control. Enzyme activity is measured as described in Magi-Galluzzi et al., 1997.

A test compound which decreases the activity of the MAP kinase phosphatase-like enzyme relative to the activity in the absence of the test compound is identified as an inhibitor of MAP kinase phosphatase-like enzyme activity.

### EXAMPLE 6

ERK Activation and Neuronal Differentiation are Differentially Sensitive to MAP Kinase Phosphatase-Like Enzyme Expression

Insights into physiological roles of MAP kinases have come from transient transfection studies where the extracellular signal regulated kinases (ERKs) activation was blocked by MAP kinase phosphatase (MKP) overexpression (60, 68, 70, 73). For example, transient expression of MKP-1 and MKP-2 into PC12 cells inhibits ERK-dependent pathways (68). However, transient transfection, which permits transcription from multiple copies of the exogenous plasmid DNA, results in levels of expression that generally exceed those reached during physiological induction of transcription. In contrast to the action of transiently transfected MKPs, physiological induction of endogenous MKP-1 and MKP-2 in PC12 cells following NGF treatment does not correlate with the inactivation of ERKs (68). These observations suggest that the specificity of MKPs actions may depend upon their level of expression as well as other factors. Less robust expression can be achieved using stable expression of transfected genes which requires chromosomal integration and selection and therefore may mimic more closely the levels reached during physiological stimulations. This example compares the effects of transient and stable expression of transfected MAP kinase phosphatase-like enzyme on signaling pathways initiated by extracellular stimuli that activate the ERK signal transduction cascade and demonstrates that ERK activation and neuronal differentiation are differentially sensitive to MAP kinase phosphatase-like expression.

Cell culture. PC12-GR5 cells are grown at 5% CO₂ in Dulbecco's modified Eagle's medium (DMEM) containing 5% fetal calf serum, 10% horse serum, and L-glutamine. Prior to drug treatments, the cells are serum starved for 24 hours with DMEM alone and are subsequently treated with 100 ng/ml NGF, 50 ng/ml EGF, 10 μM Forskolin, or 100 nM PMA.

Plasmids. Full length MAP kinase phosphatase-like enzyme cDNA is subcloned into pcDNA3 (Invitrogen) under the cytomegalovirus (CMV) promoter to generate CMV-MKPL. pcDNA3 contains the neomycin gene driven by the SV40 promoter.

Transient transfections. Sixty to eighty percent confluent cells are co-transfected using the standard calcium phosphate co-precipitation method (Gibco BRL) with the indicated combinations of the following plasmids: 10 μg of RSV-β-galactosidase, 20 μg of CMV-MKPL, 10 μg of RasV12 or 5 μg of Gal5-Elk-1 and 5 μg of 5×Gal4-E1B-luciferase. To determine the transcriptional activation of c-jun, cells are transfected with or without 1 μg of MEKK and 5 μg of both Ga14-c-jun and 5×Gal4-E1B-luciferase as indicated. The parent vector pcDNA3 is added to each set of transfections to equalize the amount of DNA the cells receive. Four hours after transfection, cells are glycerol shocked and allowed to recover in serum containing media overnight. Cells are then starved overnight in supplemented serum free medium (N2) which contains DMEM with 5 μg/ml Insulin, 100 μg/ml apo-transferrin, 30 μM sodium selenite, 100 μM putrescine, and 20 nM progesterone.

Cells are then treated with the indicated drugs for 6 hours prior to harvesting. Briefly, cells are washed twice in phosphate buffered saline (PBS), scraped in PBS, spun at low speed to collect cells, and lysed by freeze-thawing three times in 100 mM K₂PO₄, pH 7.8. The lysate is spun at high speed and the supernatant is assayed for luciferase activity using a luminometer (AutoLumat LB953).

Histological detection of β-galactosidase. The expression of β-galactosidase is used to identify transfectants within the population of differentiating cells. For counting blue cells (β-galactosidase positive) with neurites, the transfected cells are exposed to NGF for 2 days prior to fixation. PC12 cells are fixed in 2% paraformaldehyde and 0.2% glutaraldehyde for 5 minutes after which cells are washed in PBS and subjected to a β-galactosidase assay. Cells are exposed to 2 mM MgCl₂, 5 mM ferric cyanide, 5 mM ferrous cyanide, and 0.1% X-gal in PBS overnight at 37° C. Transfected cells are identified as those staining and are then counted to determine the percent of blue cells with neurites in each set of transfections. Each set of transfections is done in duplicate and 200 cells are counted for each experimental condition.

Stable transfections. PC12-GR5 cells are seeded at 3×10⁵ cells per 100 mm plate 48 hours prior to transfection. Cells are transfected with 20 μg of CMV-MKPL by calcium phosphate co-precipitation and are exposed to the precipitate for 4 hours. The cells are then glycerol shocked and allowed to recover in complete medium. Forty-eight hours later, cells are split and plated in complete medium containing 800 μg/ml G418. Stable neomycin-resistant cells were clonally isolated using cloning rings at 3 weeks post-transfection and maintained in media containing 600 μg/ml G418.

RNA Isolation, Riboprobe synthesis, and Northern blot analysis. RNA isolation using RNAzol B and MKPL riboprobe synthesis is carried out as described elsewhere (68). The c-fos transcript is detected by linearizing the 1.3 kb pGEM-c-fos plasmid with Eco RI and using SP6 RNA polymerase for antisense RNA probe synthesis in the presence of β-³²P-UTP (40 μCi/μl). Stromelysin transcripts are detected by linearizing pGEM-TR1 with Hind III and using T7 RNA polymerase to make antisense RNA transcripts. The conditions for Northern blotting using cRNA probes has been described (68). All filters are scanned and quantitated using a Molecular Dynamics PhosphorImager 445SI.

Proliferation Assay. Equal numbers of cells (2000/well) are seeded on 96-well plates. Proliferation is assessed by using the Cell Proliferation ELISA, BrdU kit (Boehringer Mannheim). Briefly, cells are labeled with 10 μM BrdU for 4 hours after which they are fixed directly on the plate. Cells are then incubated with a BrdU-antibody, washed 3 times, and incubated with substrate for 10 minutes prior to addition of the 1M H₂SO₄ stop dye. Results are quantized immediately on a ELISA reader at 450 nm. Each day represents an average of six independent wells for each of the three cell lines. Cells are also serum deprived by exposure to N2 media for 2 days prior to stimulation with serum containing media for the days indicated. Again, each day represents an average of six independent wells for each cell line.

ERK immune complex assay. Treated and untreated cells are lysed in a lysis buffer containing 10% sucrose, 1% NP-40, 20 mM Tris HCl pH 8.0, 137 mM NaCl, 10% glycerol, 2 mM EDTA, 1 mM PMSF, 1 μg/ml leupeptin, 1 mM sodium vanadate, and 10 mM sodium fluoride. The lysates are spun at low speed to remove nuclei and the supernatant is assayed for ERK activity. One hundred micrograms of total protein (as determined by Bradford Assay) is immunoprecipitated with an agarose-coupled antibody to ERK-1 (C-16) overnight at 4° C. The immunoprecipitated ERK-1 is washed 3 times in lysis buffer and is assayed for kinase activity by incubating with 10 μg myelin basic protein (MBP) and 10 μCi β³² P-ATP in 50 μl of buffer containing 80 mM Hepes pH 7.4, 80 mM MgCl₂, 0.1 mM ATP, 2 mM sodium vanadate, and 20 mM sodium fluoride for 30 minutes at 30° C. Reactions are terminated by the addition of 50 μl of Laemmli sample buffer and analyzed by SDS-PAGE. Quantitations are performed by scanning the gel using a Phosphorlmager.

JNK immune complex assay. Treated and untreated cells are lysed in a lysis buffer containing 20 mM Hepes-KOH pH 7.4, 2 mM EGTA, 50 mM β-glycerophosphate, 10% glycerol, 1% Triton X-100, 1 mM DTT, 1 mM sodium vanadate, 0.4 mM PMSF, 0.5 μg/ml Aprotinin, and 0.5 μg/ml leupeptin. The lysates are spun at low speed to remove nuclei and the supernatant is assayed for JKN activity. One hundred micrograms of total protein (as determined by Bradford Assay) is immuno-precipitated with an agarose-coupled antibody to JNK-1 (FL) overnight at 4° C. The immunoprecipitated JNK-1 is washed 3 times in each of 3 buffers (lysis buffer, LiCl buffer {500 mM LiCl, 100 mM Tris-HCl pH 7.6, 1 mM DTT, and 0.1% Triton X-100}, and Assay buffer {20 mM MOPS pH 7.2, 10 MM MgCl₂, 2 mM EGTA, 1 mM DTT, and 0.1% Triton X-100}) and is assayed for kinase activity by incubating with 3 μg Gst-c-jun and 1 μCi β³² P-ATP in assay buffer for 30 minutes at 30° C. Reactions are terminated by the addition of 50 μl of Laemmli sample buffer and analyzed by SDS-PAGE. Quantitations are performed by scanning the gel using a Phosphorlmager.

Morphological determination. Cells are grown on Primaria plates and are serum starved in N2 medium for 24 hours (control) and subsequently treated with NGF in N2 media. Cells are washed twice with PBS and fixed in 4% paraformaldehyde and 0.1% glutaraldehyde for 5 minutes after which they are washed in PBS. Cells are photographed at 70×magnification with a Leitz Dialux 22 EB.

Results. Transient transfection of MAP kinase phosphatase-like enzyme blocks neurite outgrowth in PC12 cells. To study the action of MAP kinase phosphatase-like enzyme in governing cellular differentiation, PC12 cells are transiently transfected with expression vectors carrying the coding regions of MAP kinase phosphatase-like enzyme or with the control vector. A gene encoding β-galactosidase also is transiently transfected into the cells to provide a marker for transfected cells following histological staining for β-galactosidase activity. One set of transfections contains a vector encoding active Ras (RasV12) along with the other plasmids, while the other set is treated with NGF for 48 hours prior to performing the β-galactosidase staining. Both NGF treated cells and cells receiving the RasV12 plasmid develop neurites in 40-60% of the β-galactosidase positive cells. However, following transient transfection of MAP kinase phosphatase-like enzyme, only 10-20% of the β-galactosidase positive cells show neurites.

These results demonstrate that MAP kinase phosphatase-like enzyme can inhibit neuronal differentiation when transiently overexpressed, as has been observed for MKP-1 (73). In contrast, under more physiological conditions, the induction of endogenous MKP-1 and MKP-2 mRNA expression by NGF does not block neuronal differentiation (68). It is important, therefore, to examine PC12 cells where the overexpression of MAP kinase phosphatase-like enzyme is maintained at more physiological levels. This can be achieved by examining stable PC12-derived cell lines that express MAP kinase phosphatase-like enzyme to levels that are similar to that achieved following induction of other MKPs by physiological agents.

### EXAMPLE 7

ERK Activity is Reduced in AMP Kinase Phosphatase-Like Enzyme Overexpressing Cells.

Generation of MAP kinase phosphatase-like enzyme overexpressing stable cell lines. To investigate the consequences of limited overexpression of MAP kinase phosphatase-like enzyme, clonal isolates that stably express the enzyme are generated. PC12 cells are transfected with MKPL cDNA under the control of the CMV promoter and selected using neomycin. The expression of the transgenes is only 2-3 fold over basal in every positive clone analyzed. This level of expression is similar to levels achieved following induction by NGF and EGF (68). Therefore, these clonal lines provide a model for the action of physiological levels of MAP kinase phosphatase-like enzyme expression.

ERK activity is reduced in MAP kinase phosphatase-like enzyme overexpressing cells. Growth factor, hormone, and phorbol ester stimulation of PC12 cells have been known to activate the MAP kinase pathway and to stimulate the enzymatic activity of ERK-1 (61, 62, 65, 69, 71). The enzymatic activity of ERK-1 in wild type cells is compared with that in MAP kinase phosphatase-like enzyme overexpressing cells treated with these agents. PC12 cells treated for 10 minutes with mitogenic agents such as EGF, differentiating agents such as NGF and forskolin, and the tumor promoter phorbol 12-myristate 132-acetate (PMA), produce a robust activation of ERK-1 as measured by an immune complex activity assay. MAP kinase phosphatase-like enzyme overexpressing clones are then treated with the same agents for the identical times. These clones show a dramatic reduction in the ability of growth factors and hormones to activate ERK-1. Quantitation of the immune complex assays shows that modest overexpression of MAP kinase phosphatase-like enzyme in PC12 cells inhibits growth factor- and hormone-induced activation of ERKs 80-90% cells compared to the fold activation seen in wild type PC12 cells. The basal ERK activity also appears to be lower in these overexpression cells as compared to wild type cells.

### EXAMPLE 8

MAP Kinase Phosphatase-Like Enzyme Overexpression Cells Exhibit Reduced Activation of ERK-Responsive Transcription

To determine if the MAP kinase phosphatase-like enzyme-induced reduction in ERK-activation by growth factors and other agents leads to changes in gene expression, the ability of MAP kinase phosphatase-like enzyme overexpression cells to activate transcription of an ERK-dependent gene through the transcriptional activator, Elk-1 is examined. Several studies have shown that ERK phosphorylation sites in the carboxyl terminal transcriptional activation domain of Elk-1 are sufficient to allow transcription response to growth factors (63).

Cells are transiently transfected with the chimeric reporter genes Gal4-Elk-1 and 5XGal4-E1B-luciferase and the next day are treated with EGF or NGF for 6 hours prior to harvesting and performing luciferase assays. These agents are thought to activate Elk-1 through their action on ERKs. The physiological activation of ERKs results in increased luciferase activity. The activation of Elk-1 transcription activational activity by the ERK cascade stimulators (EGF and NGF) is reduced in MAP kinase phosphatase-like overexpressing cells. Although Elk-1 can be activated by JNKs as well as ERKs (72), neither agent activates JNKs significantly in either wild type or overexpressing cells. Therefore, Elk-1 activation by these agents reflects ERK activation rather than JNK activation. The reduced EGF and NGF-induced ERK activity in these cells is therefore likely responsible for the reduction in ERK-dependent gene expression.

### EXAMPLE 9

Effect of MAP Kinase Phosphatase-Like Enzyme Overexpression on Proliferation and Differentiation

Previous reports suggest that the sustained activation of ERKs by NGF is required for PC12 cell differentiation whereas the transient activation of ERKs by EGF is required for proliferation (57, 66). To examine the biological consequences of MAP kinase phosphatase-like enzyme overexpression and reduced growth factor-inducible ERK activation, the proliferation rate of overexpressing cells can be measured. The proliferation rate of parental PC 12 cells growing in serum is higher than that of the overexpressing cells. When the cells are partially synchronized by serum starvation for 2 days and then stimulated with serum, overexpressing cells are delayed in their entry into the cell cycle. These results suggest that the reduced growth factor-stimulated ERK activity is associated with a reduction in proliferation in MAP kinase phosphatase-like enzyme overexpressing cells.

### EXAMPLE 10

Quantitative Expression Profiling of MAP Kinase Phosphatase-Like Enzyme

Expression profiling is based on a quantitative polymerase chain reaction (PCR) analysis, also called kinetic analysis, first described in Higuchi et al., 1992 and Higuchi et al., 1993. The principle is that at any given cycle within the exponential phase of PCR, the amount of product is proportional to the initial number of template copies. Using this technique, the expression levels of particular genes, which are transcribed from the chromosomes as messenger RNA (mRNA), are measured by first making a DNA copy (cDNA) of the mRNA, and then performing quantitative PCR on the cDNA, a method called quantitative reverse transcription-polymerase chain reaction (quantitative RT-PCR).

Quantitative RT-PCR analysis of RNA from different human tissues was performed to investigate the tissue distribution of MAP kinase phosphatase-like mRNA. In most cases, 25 μg of total RNA from various tissues (including Human Total RNA Panel I-V, Clontech Laboratories, Palo Alto, Calif., USA) was used as a template to synthsize first-strand cDNA using the SUPERSCRIPT™ First-Strand Synthesis System for RT-PCR (Life Technologies, Rockville, Md., USA). First-strand cDNA synthesis was carried out according to the manufacturer's protocol using oligo (dT) to hybridize to the 3′ poly A tails of mRNA and prime the synthesis reaction. Approximately 10 ng of the first-strand cDNA was then used as template in a polymerase chain reaction. In other cases, 10 ng of commercially available cDNAs (Human Immune System MTC Panel and Human Blood Fractions MTC Panel, Clontech Laboratories, Palo Alto, Calif., USA) were used as template in a polymerase chain reaction. The polymerase chain reaction was performed in a LightCycler (Roche Molecular Biochemicals, Indianapolis, Ind., USA), in the presence of the DNA-binding fluorescent dye SYBR Green I which binds to the minor groove of the DNA double helix, produced only when double-stranded DNA is successfully synthesized in the reaction (Morrison et al., 1998). Upon binding to double-stranded DNA, SYBR Green I emits light that can be quantitatively measured by the LightCycler machine. The polymerase chain reaction was carried out using oligonucleotide primers LBRI_{—}184-R4 (5′-ATTTCCTCTACGCCCAGGTTCC-3′) and LBRI_{—}184-R4 (5′-TGTAGTGACAGGGGTGGGAGGT-3′) and measurements of the intensity of emitted light were taken following each cycle of the reaction when the reaction had reached a temperature of 87 degrees C. Intensities of emitted light were converted into copy numbers of the gene transcript per nanogram of template cDNA by comparison with simultaneously reacted standards of known concentration. To correct for differences in mRNA transcription levels per cell in the various tissue types, a normalization procedure was performed using similarly calculated expression levels in the various tissues of five different housekeeping genes: glyceraldehyde-3-phosphatase (G3PDH), hypoxanthine guanine phophoribosyl transferase (HPRT), beta-actin, porphobilinogen deaminase (PBGD), and beta-2-microglobulin. The level of housekeeping gene expression is considered to be relatively constant for all tissues (Adams et al., 1993, Adams et al., 1995, Liew et al., 1994) and therefore can be used as a gauge to approximate relative numbers of cells per .mu.g of total RNA used in the cDNA synthesis step. Except for the use of a slightly different set of housekeeping genes and the use of the LightCycler system to measure expression levels, the normalization procedure was similar to that described in the RNA Master Blot User Manual, Apendix C (1997, Clontech Laboratories, Palo Alto, Calif., USA). In brief, expression levels of the five housekeeping genes in all tissue samples were measured in three independent reactions per gene using the LightCycler and a constant amount (25 μg) of starting RNA. The calculated copy numbers for each gene, derived from comparison with simultaneously reacted standards of known concentrations, were recorded and the mean number of copies of each gene in all tissue samples was determined. Then for each tissue sample, the expression of each housekeeping gene relative to the mean was calculated, and the average of these values over the five housekeeping genes was found. A normalization factor for each tissue was then calculated by dividing the final value for one of the tissues arbitrarily selected as a standard by the corresponding value for each of the tissues. To normalize an experimentally obtained value for the expression of a particular gene in a tissue sample, the obtained value was multiplied by the normalization factor for the tissue tested. This normalization method was used for all tissues except those derived from the Human Blood Fractions MTC Panel, which showed dramatic variation in some housekeeping genes depending on whether the tissue had been activated or not. In these tissues, normalization was carried out with a single housekeeping gene, beta-2-microglobulin.

Results are shown in FIGS. 17 and 18, showing the experimentally obtained copy numbers of mRNA per 10 ng of first-strand cDNA on the left and the normalized values on the right. RNAs used for the cDNA synthesis, along with their supplier and catalog numbers are shown in Tables 1 and 2.

Higuchi, R., Dollinger, G., Walsh, P. S. and Griffith, R. (1992) Simultaneous amplification and detection of specific DNA sequences. BioTechnology 10:413-417.

Higuchi, R., Fockler, C., Dollinger, G. and Watson, R. (1993) Kinetic PCR analysis: real-time monitoring of DNA amplification reactions. BioTechnology 11:1026-1030.

T. B. Morrison, J. J. Weis & C. T. Wittwer .(1998) Quantification of low-copy transcripts by continuous SYBR Green I monitoring during amplification. Biotechniques 24:954-962.

Adams, M. D., Kerlavage, A. R., Fields, C. & Venter, C. (1993) 3,400 new expressed sequence tags identify diversity of transcripts in human brain. Nature Genet. 4:256-265.

Adams, M. D., et al. (1995) Initial assessment of human gene diversity and expression patterns based upon 83 million nucleotides of cDNA sequence. Nature 377 supp:3-174.

Liew, C. C., Hwang, D. M., Fung, Y. W., Laurenson, C., Cukerman, E., Tsui, S. & Lee, C. Y. (1994) A catalog of genes in the cardiovascular system as identified by expressed sequence tags. Proc. Natl. Acad. Sci. USA 91:10145-10649.

TABLE 1
Whole-body-screen tissues

| Tissue | Supplier | Panel name and catalog number |
|---|---|---|
| 1. brain | Clontech | Human Total RNA Panel I, K4000-1 |
| 2. heart | Clontech | Human Total RNA Panel I, K4000-1 |
| 3. kidney | Clontech | Human Total RNA Panel I, K4000-1 |
| 4. liver | Clontech | Human Total RNA Panel I, K4000-1 |
| 5. lung | Clontech | Human Total RNA Panel I, K4000-1 |
| 6. trachea | Clontech | Human Total RNA Panel I, K4000-1 |
| 7. bone marrow | Clontech | Human Total RNA Panel II, K4001-1 |
| 8. colon | Clontech | Human Total RNA Panel II, K4001-1 |
| 9. small intestine | Clontech | Human Total RNA Panel II, K4001-1 |
| 10. spleen | Clontech | Human Total RNA Panel II, K4001-1 |
| 11. stomach | Clontech | Human Total RNA Panel II, K4001-1 |
| 12. thymus | Clontech | Human Total RNA Panel II, K4001-1 |
| 13. mammary gland | Clontech | Human Total RNA Panel III, K4002-1 |
| 14. skeletal muscle | Clontech | Human Total RNA Panel III, K4002-1 |
| 15. prostate | Clontech | Human Total RNA Panel III, K4002-1 |
| 16. testis | Clontech | Human Total RNA Panel III, K4002-1 |
| 17. uterus | Clontech | Human Total RNA Panel III, K4002-1 |
| 18. cerebellum | Clontech | Human Total RNA Panel IV, K4003-1 |
| 19. fetal brain | Clontech | Human Total RNA Panel IV, K4003-1 |
| 20. fetal liver | Clontech | Human Total RNA Panel IV, K4003-1 |
| 21. spinal cord | Clontech | Human Total RNA Panel IV, K4003-1 |
| 22. placenta | Clontech | Human Total RNA Panel IV, K4003-1 |
| 23. adrenal gland | Clontech | Human Total RNA Panel V, K4004-1 |
| 24. pancreas | Clontech | Human Total RNA Panel V, K4004-1 |
| 25. salivary gland | Clontech | Human Total RNA Panel V, K4004-1 |
| 26. thyroid | Clontech | Human Total RNA Panel V, K4004-1 |

TABLE 2
Blood/lung-screen tissues

| Tissue | | Supplier | Panel name and catalog number |
|---|---|---|---|
| 1. | lymph node | Clontech | Human Immune System MTC Panel, K1426-1 |
| 2. | peripheral blood leukocytes | Clontech | Human Immune System MTC Panel, K1426-1 |
| 3. | tonsil | Clontech | Human Immune System MTC Panel, K1426-1 |
| 4. | peripheral blood mononuclear | Clontech | Human Blood Fractions MTC Panel, K1428-1 |
| | cells | | |
| 5. | peripheral blood mononuclear | Clontech | Human Blood Fractions MTC Panel, K1428-1 |
| | cells - activated | | |
| 6. | T-cell (CD8+) | Clontech | Human Blood Fractions MTC Panel, K1428-1 |
| 7. | T-cell (CD8+) - activated | Clontech | Human Blood Fractions MTC Panel, K1428-1 |
| 8. | T-cell (CD4+) | Clontech | Human Blood Fractions MTC Panel, K1428-1 |
| 9. | T-cell (CD4+) - activated | Clontech | Human Blood Fractions MTC Panel, K1428-1 |
| 10. | B-cell (CD19+) | Clontech | Human Blood Fractions MTC Panel, K1428-1 |
| 11. | B-cell (CD19+) - activated | Clontech | Human Blood Fractions MTC Panel, K1428-1 |
| 12. | Monocytes (CD14+) | Clontech | Human Blood Fractions MTC Panel, K1428-1 |
| 13. | Th1 clone | In-house | |
| 14. | Th2 clone | In-house | |
| 15. | neutrophil | In-house | |
| 16. | natural killer cells | In-house | |
| 17. | lymphokine-activated killer | In-house | |
| | cells | | |
| 18. | natural killer cells | In-house | |
| 19. | interleukin-2-activated natural | In-house | |
| | killer cells | | |
| 20. | Normal Bronchial/Tracheal | In-house | |
| | Epithelial Cells | | |
| 21. | Normal Bronchial/Tracheal | In-house | |
| | smooth muscle cell | | |
| 22. | Normal lung fibroblast | In-house | |
| 23. | Microvascular Endothelial cell | In-house | |
| 24. | RAMOS | In-house | |
| 25. | Jurkat | In-house | |
| 26. | HEK293 | In-house | |

### REFERENCES

1. Pages, G., Lenormand, P., L'Allenmain, G., Chambard, J.-C., Meloche, S., and Pouyssegur, J. (1993) Proc. Nati. Acad. Sci. U.S.A. 90, 8319-8323.

2. Blenis, J. (1993) Proc. Natl. Acad. Sci. U.S.A. 90, 5889-5892.

3. Cowley, S., Paterson, H., Kemp, P., and Marshall, C. J. (1994) Cell 77, 841-852.

4. Chao, M. V. (1992) Cell 68, 995-997.

5. Nguyen, T. T., Scimeca, J.-C., Filloux, C., Peraldi, P., Carpentier, J.-L., and VanObberghen, E. (1993) J. Biol. Chem. 268, 9803-9810.

6. Wood, K. W., Sarnecki, C., Roberts, T. M., and Blenis, K. (1992) Cell 68, 1041-150.

7. Alema, S., Casalbore, P., Agostini, E., and Tato, F. (1985) Nature 316, 557-559.

8. Thomas, S. M., DeMarco, M., D'Arcangelo, G., Halegoua, S., and Brugge, J. S. (1992) Cell 68,1031-1040.

9. Thomas, S. M., Hayes, M., D'Arcangelo, G., Armstrong, R. C., Meyer, B. D., Zilberstein, A., Brugge, J. S., and Halegoua, S. (1991) Mol. Cell. Biol. 11, 4739-4750.

10. Lloyd, E. D., and Wooten, M. W. (1992) J. Nerochem. 59,1099-1109.

11. Crews, C. M., and Erikson, R. L. (1993) Cell 74, 215-217.

12. Zheng, C.-F., and Guan, K.-L. (1993) J. Biol. Chem. 268, 23933-23939.

13. Sun, H., Charles, C. H., Lau, L. F., and Tonks, N. K. (1993) Cell 75, 487-493.

14. Allessi, D. R., Smythe, C., and Keyse, S. M. (1993) Oncogene 8, 2015-2020.

15. Ward, Y., Gupta, S., Jensen, P., Wartmann, M., Davis, R. J., and Kelly, K. (1994) Nature 367, 651-654.

16. Zheng, C.-F., and Guan, K.-L. (1993) J. Biol. Chem. 268, 16116-16119.

17. Traverse, S., Gomez, N., Paterson, H., Marshall, C., and Cohen, P. (1992) Biochem. J. 288, 351-355.

18. Kumagai, A., and Dunphy, W. G. (1992) Cell 70, 139-151.

19. Charles, C. H., Abler, A. S., and Lau, L. C. (1992) Oncogene 7, 187-190.

20. Doi, K., Gamer, A., Ammerer, G., Errede, B., Shinkawa, H., Sugimoto, K., and Matsumoto, K. (1994) EMBO J. 13, 61-70.

21. Guan, K., Haun, R. S., Watson, S. J., Geahlen, R. L., and Dixon, J. E. (1990) Proc. Natl. Acad. Sci. U.S.A. 87, 1501-1505.

22. Keyse, S. M., and Emslie, E. A. (1992) Nature 359, 644-647.

23. Rohan, P. J., Davis, P., Moskaluk, C. A., Kearns, M., Krutzch, H., Siebenlist, and Kelly, K. (1993) Science 259, 1763-1766.

24. Ishibashi, T., Bottaro, D. P., Michieli, P., Kelley, C. A., and Aaronson, S. A. (1994) J. Biol. Chem. 269, 29897-29902.

25. Sun, H., Tonks, N. K., and Bar-Sagi, D. (1994) Science 266, 285-288.

26. Noguchi, T., Metz, R., Chen, L., Mattei, M.-G., Carrasco, D., and Bravo, R. (1993) Mol. Cell. Biol. 13, 5195-5205.

27. Boulton, T. G., Nye, S. H., Robbins, D. J., Ip, N. Y., Radziejewska, E., Morgenbesser, S. D., DePinho, R. A., N, P., Cobb, M. H., and Yancopoulos, G. D. (1991) Cell 65, 663-675.

28. Greene, L. A., and Tischler, A. S. (1982) Adv. Cell. Neurobiol. 3, 373-414.

29. Thomas, K. R., Musci, T. S., Neumann, P. E., and Capecehi, M. R. (1991) Cell 67, 969-976.

57. Cowley, S., Paterson, H., Kemp, P. and Marshall, C. J. (1994) Cell 77, 841-852.

60. Fukuda, M., Gotoh, Y., Tachibana, T., Dell, K., Hattori, S., Yoneda, Y. and Nishida, E. Oncogene 11, 239-344.

61. Hill, C. S. and Treisman, R. (1995) Cell 80, 199-211.

62. Jaiswal, R. K., Murphy, M. B. and Landreth, G. E. (1993) J. Biol. Chem. 268, 7055-7063.

63. Janknecht, R., Ernst, W. H., Pingound, V. and Nordheim, A. (1993) EMBO J. 12, 5097-5104.

65. Lloyd, E. D., and M. W. Wooten (1992) J. Nerochem. 59,1099-1109.

66. Marshall, C. J. (1995) Cell 80, 179-185.

68. Misra-Press, A., C. S. Rim, H. Yao, M. S. Roberson, and P. J. S. Stork (1995) J. Biol. Chem. 270, 14587-14596.

69. Qui, M.-S., and S. H. Green (1991) Neuron 7, 937-946.

70. Roberson, M. S., A. Misra-Press, M. E. Laurance, P. J. S. Stork, and R. A. Maurer (1995) Mol. Cell Biol. 17, 3531-3539.

71. Rosen, L. B., D. D. Ginty, M. J. Weber, and M. E. Greenberg (1994) Neuron 12, 1207-1221.

73. Yao, H., K. Labudda, C. Rim, P. Capodieci, M. Loda, and P. J. S. Stork (1995) J. Biol. Chem. 270, 20748-20753.

79. Liu, Y., Gorospe, M., Yang, C. and Halbrook, N. J. (1995) J. Biol. Chem. 270, 8377-8380.

84. Thompson, C. B. (1995) Science 267, 1456-1462.

85. Kharbanda, S., Saleem, A., Shafman, T., Emoto, Y., Taneja, N., Rubin, E., Weichselbaum, R., Woodgett, J., Avruch, J., Kyriakis, J. and Kufe, D. (1995) J. Biol. Chem. 270, 18871-18874.

86. Kerr, J. F. R., Winterford, C. M. and Harmon, B. V. (1994) Cancer 73, 2013-2026.

1. An isolated polynucleotide encoding a MAP kinase phosphatase-like enzyme polypeptide and being selected from the group consisting of:
a) a polynucleotide encoding a MAP kinase phosphatase-like enzyme polypeptide comprising an amino acid sequence selected form the group consisting of:
amino acid sequences which are at least about 50% identical to
the amino acid sequence shown in SEQ ID NO: 2;
the amino acid sequence shown in SEQ ID NO: 2;
amino acid sequences which are at least about 50% identical to
the amino acid sequence shown in SEQ ID NO: 11; and
the amino acid sequence shown in SEQ ID NO:11.
b) a polynucleotide comprising the sequence of SEQ ID NO: 1 or 10;
c) a polynucleotide which hybridizes under stringent conditions to a polynucleotide specified in (a) and (b);
d) a polynucleotide the sequence of which deviates from the polynucleotide sequences specified in (a) to (c) due to the degeneration of the genetic code; and
e) a polynucleotide which represents a fragment, derivative or allelic variation of a polynucleotide sequence specified in (a to (d).

2. An expression vector containing any polynucleotide of claim 1.

3. A host cell containing the expression vector of claim 2.

4. A substantially purified MAP kinase phosphatase-like enzyme polypeptide encoded by a polynucleotide of claim 1.

5. A method for producing a MAP kinase phosphatase-like enzyme polypeptide, wherein the method comprises the following steps:
a) culturing the host cell of claim 3 under conditions suitable for the expression of the MAP kinase phosphatase-like enzyme polypeptide; and
b) recovering the MAP kinase phosphatase-like enzyme polypeptide from the host cell culture.

6. A method for detection of a polynucleotide encoding a MAP kinase phosphatase-like enzyme polypeptide in a biological sample comprising the following steps:
a) hybridizing any polynucleotide of claim 1 to a nucleic acid material of a biological sample, thereby forming a hybridization complex; and
b) detecting said hybridization complex.

7. The method of claim 6, wherein before hybridization, the nucleic acid material of the biological sample is amplified.

8. A method for the detection of a polynucleotide of claim 1 or a MAP kinase phosphatase-like enzyme polypeptide of claim 4 comprising the steps of:
contacting a biological sample with a reagent which specifically interacts with the polynucleotide or the MAP kinase phosphatase-like enzyme polypeptide.

9. A diagnostic kit for conducting the method of any one of claims 6 to 8.

10. A method of screening for agents which decrease the activity of a MAP kinase phosphatase-like enzyme, comprising the steps of:
contacting a test compound with any MAP kinase phosphatase-like enzyme polypeptide encoded by any polynucleotide of claim 1;
detecting binding of the test compound to the MAP kinase phosphatase-like enzyme polypeptide, wherein a test compound which binds to the polypeptide is identified as a potential therapeutic agent for decreasing the activity of a MAP kinase phosphatase-like enzyme.

11. A method of screening for agents which regulate the activity of a MAP kinase phosphatase-like enzyme, comprising the steps of:
contacting a test compound with a MAP kinase phosphatase-like enzyme polypeptide encoded by any polynucleotide of claim 1; and
detecting a MAP kinase phosphatase-like enzyme activity of the polypeptide, wherein a test compound which increases the MAP kinase phosphatase-like enzyme activity is identified as a potential therapeutic agent for increasing the activity of the MAP kinase phosphatase-like enzyme, and wherein a test compound which decreases the MAP kinase phosphatase-like enzyme activity of the polypeptide is identified as a potential therapeutic agent for decreasing the activity of the MAP kinase phosphatase-like enzyme.

12. A method of screening for agents which decrease the activity of a MAP kinase phosphatase-like enzyme, comprising the steps of:
contacting a test compound with any polynucleotide of claim 1 and detecting binding of the test compound to the polynucleotide, wherein a test compound which binds to the polynucleotide is identified as a potential therapeutic agent for decreasing the activity of MAP kinase phosphatase-like enzyme.

13. A method of reducing the activity of MAP kinase phosphatase-like enzyme, comprising the steps of:
contacting a cell with a reagent which specifically binds to any polynucleotide of claim 1 or any MAP kinase phosphatase-like enzyme polypeptide of claim 4, whereby the activity of MAP kinase phosphatase-like enzyme is reduced.

14. A reagent that modulates the activity of a MAP kinase phosphatase-like enzyme polypeptide or a polynucleotide wherein said reagent is identified by the method of any of the claim 10 to 12.

15. A pharmaceutical composition, comprising:
the expression vector of claim 2 or the reagent of claim 14 and a pharmaceutically acceptable carrier.

16. Use of the pharmaceutical composition of claim 15 for modulating the activity of a MAP kinase phosphatase-like enzyme in a disease.

17. Use of claim 16 wherein the disease is asthma, a CNS disorder, diabetes, obesity, chronic obstructive pulmonary disease, cancer or a cardiovascular disease.

18. A cDNA encoding a polypeptide comprising the amino acid sequence shown in SEQ ID NO:2 or 11.

19. The cDNA of claim 18 which comprises SEQ ID NO:1 or 10.

20. The cDNA of claim 18 which consists of SEQ ID NO:1 or 10.

21. An expression vector comprising a polynucleotide which encodes a polypeptide comprising the amino acid sequence shown in SEQ ID NO:2 or 11.

22. The expression vector of claim 21 wherein the polynucleotide consists of SEQ ID NO:1 or 10.

23. A host cell comprising an expression vector which encodes a polypeptide comprising the amino acid sequence shown in SEQ ID NO:2 or 11.

24. The host cell of claim 23 wherein the polynucleotide consists of SEQ ID NO:1 or 10.

25. A purified polypeptide comprising the amino acid sequence shown in SEQ ID NO:2 or 11.

26. The purified polypeptide of claim 25 which consists of the amino acid sequence shown in SEQ ID NO:2 or 11.

27. A fusion protein comprising a polypeptide having the amino acid sequence shown in SEQ ID NO:2 or 11.

28. A method of producing a polypeptide comprising the amino acid sequence shown in SEQ ID NO:2 or 11, comprising the steps of:
culturing a host cell comprising an expression vector which encodes the polypeptide under conditions whereby the polypeptide is expressed; and isolating the polypeptide.

29. The method of claim 28 wherein the expression vector comprises SEQ ID NO:1 or 10.

30. A method of detecting a coding sequence for a polypeptide comprising the amino acid sequence shown in SEQ ID NO:2 or 11, comprising the steps of:
hybridizing a polynucleotide comprising 11 contiguous nucleotides of SEQ ID NO:1 or 10 to nucleic acid material of a biological sample, thereby forming a hybridization complex; and
detecting the hybridization complex.

31. The method of claim 30 further comprising the step of amplifying the nucleic acid material before the step of hybridizing.

32. A kit for detecting a coding sequence for a polypeptide comprising the amino acid sequence shown in SEQ ID NO:2 or 11, comprising:
a polynucleotide comprising 11 contiguous nucleotides of SEQ ID NO:1 or 10; and
instructions for the method of claim 30.

33. A method of detecting a polypeptide comprising the amino acid sequence shown in SEQ ID NO:2 or 11, comprising the steps of:
contacting a biological sample with a reagent that specifically binds to the polypeptide to form a reagent-polypeptide complex; and
detecting the reagent-polypeptide complex.

34. The method of claim 33 wherein the reagent is an antibody.

35. A kit for detecting a polypeptide comprising the amino acid sequence shown in SEQ ID NO:2 or 11, comprising:
an antibody which specifically binds to the polypeptide; and
instructions for the method of claim 33.

36. A method of screening for agents which can modulate the activity of a human MAP kinase phosphatase-like enzyme, comprising the steps of:
contacting a test compound with a polypeptide comprising an amino acid sequence selected from the group consisting of: (1) amino acid sequences which are at least about 50% identical to the amino acid sequence shown in SEQ ID NO:2 or 11 and (2) the amino acid sequence shown in SEQ ID NO:2 or 11; and
detecting binding of the test compound to the polypeptide, wherein a test compound which binds to the polypeptide is identified as a potential agent for regulating activity of the human MAP kinase phosphatase-like enzyme.

37. The method of claim 36 wherein the step of contacting is in a cell.

38. The method of claim 36 wherein the cell is in vitro.

39. The method of claim 36 wherein the step of contacting is in a cell-free system.

40. The method of claim 36 wherein the polypeptide comprises a detectable label.

41. The method of claim 36 wherein the test compound comprises a detectable label.

42. The method of claim 36 wherein the test compound displaces a labeled ligand which is bound to the polypeptide.

43. The method of claim 36 wherein the polypeptide is bound to a solid support.

44. The method of claim 36 wherein the test compound is bound to a solid support.

45. A method of screening for agents which modulate an activity of a human MAP kinase phosphatase-like enzyme, comprising the steps of:
contacting a test compound with a polypeptide comprising an amino acid sequence selected from the group consisting of: (1) amino acid sequences which are at least about 50% identical to the amino acid sequence shown in SEQ ID NO:2 or 11 and (2) the amino acid sequence shown in SEQ ID NO:2 or 11; and
detecting an activity of the polypeptide, wherein a test compound which increases the activity of the polypeptide is identified as a potential agent for increasing the activity of the human MAP kinase phosphatase-like enzyme, and wherein a test compound which decreases the activity of the polypeptide is identified as a potential agent for decreasing the activity of the human MAP kinase phosphatase-like enzyme.

46. The method of claim 45 wherein the step of contacting is in a cell.

47. The method of claim 45 wherein the cell is in vitro.

48. The method of claim 45 wherein the step of contacting is in a cell-free system.

49. A method of screening for agents which modulate an activity of a human MAP kinase phosphatase-like enzyme, comprising the steps of:
contacting a test compound with a product encoded by a polynucleotide which comprises the nucleotide sequence shown in SEQ ID NO:1 or 10; and
detecting binding of the test compound to the product, wherein a test compound which binds to the product is identified as a potential agent for regulating the activity of the human MAP kinase phosphatase-like enzyme.

50. The method of claim 49 wherein the product is a polypeptide.

51. The method of claim 49 wherein the product is RNA.

52. A method of reducing activity of a human MAP kinase phosphatase-like enzyme, comprising the step of:
contacting a cell with a reagent which specifically binds to a product encoded by a polynucleotide comprising the nucleotide sequence shown in SEQ ID NO:1 or 10, whereby the activity of a human MAP kinase phosphatase-like enzyme is reduced.

53. The method of claim 52 wherein the product is a polypeptide.

54. The method of claim 53 wherein the reagent is an antibody.

55. The method of claim 52 wherein the product is RNA.

56. The method of claim 55 wherein the reagent is an antisense oligonucleotide.

57. The method of claim 56 wherein the reagent is a ribozyme.

58. The method of claim 52 wherein the cell is in vitro.

59. The method of claim 52 wherein the cell is in vivo.

60. A pharmaceutical composition, comprising:
a reagent which specifically binds to a polypeptide comprising the amino acid sequence shown in SEQ ID NO:2 or 11; and
a pharmaceutically acceptable carrier.

61. The pharmaceutical composition of claim 60 wherein the reagent is an antibody.

62. A pharmaceutical composition, comprising:
a reagent which specifically binds to a product of a polynucleotide comprising the nucleotide sequence shown in SEQ ID NO:1 or 10; and
a pharmaceutically acceptable carrier.

63. The pharmaceutical composition of claim 62 wherein the reagent is a ribozyme.

64. The pharmaceutical composition of claim 62 wherein the reagent is an antisense oligonucleotide.

65. The pharmaceutical composition of claim 62 wherein the reagent is an antibody.

66. A pharmaceutical composition, comprising:
an expression vector encoding a polypeptide comprising the amino acid sequence shown in SEQ ID NO:2 or 11; and
a pharmaceutically acceptable carrier.

67. The pharmaceutical composition of claim 66 wherein the expression vector comprises SEQ ID NO:1 or 10.

68. A method of treating a MAP kinase phosphatase-like enzyme dysfunction related disease, wherein the disease is selected from asthma, a CNS disorder, diabetes, obesity, chronic obstructive pulmonary disease, cancer or a cardiovascular disease, comprising the step of:
administering to a patient in need thereof a therapeutically effective dose of a reagent that modulates a function of a human MAP kinase phosphatase-like enzyme, whereby symptoms of the MAP kinase phosphatase-like enzyme dysfunction related disease are ameliorated.

69. The method of claim 68 wherein the reagent is identified by the method of claim 36.

70. The method of claim 68 wherein the reagent is identified by the method of claim 45.

71. The method of claim 68 wherein the reagent is identified by the method of claim 49.
